(19) United States
(12) Patent Application Publication
(10) Pub. No.: US 2003/0219454 A1
(43) Pub. Date: Nov. 27, 2003

(54) Haemagglutinin antigen

(76) Inventors: Tamsin Deborah Terry, Queensland (AU); Hsing-Ju Tseng, Queensland (AU); Rhonda Ivy Hobb, Decatur, GA (US); Michael Paul Jennings, Queensland (AU); John Downes, Queensland (AU)

Correspondence Address: HELLER EHRMAN WHITE & MCAULIFFE LLP, 1666 K STREET,NW, SUITE 300, WASHINGTON, DC, 20006, US

(21) Appl. No.: 10/336,840

(22) Filed: Jan. 6, 2003

Related U.S. Application Data
(63) Continuation of application No. PCT/AU01/00822, filed on Jul. 6, 2001.

(51) Int. Cl. A61K 39/02; C12N 9/50; C07H 21/04; C12P 21/02; C12N 1/21; C12N 15/74

(52) U.S. Cl. 424/190.1; 435/69.3; 435/252.3; 435/219; 435/320.1; 536/23.2

(57) ABSTRACT

Haemagglutinin polypeptides and encoding nucleic acids are isolated from eleven strains of Haemophilus paragallinarum. The haemagglutinin polypeptides are useful in vaccines for immunization against infectious coryza in chickens, as are the encoding nucleic acids when expressed in attenuated bacteria. Also provided are methods of use of the haemagglutinin polypeptides and nucleic acids for detection and diagnosis of, and immunization against, infectious coryza in chickens.

### FIELD OF THE INVENTION

THIS INVENTION relates to haemagglutinin polypeptides of Haemophilus paragallinarum and nucleic acids encoding same. More particularly, this invention relates to diagnosis of, and immunization against, infectious coryza in chickens using a haemagglutinin polypeptide and/or encoding nucleic acid, although without being limited thereto.

### BACKGROUND OF THE INVENTION

Haemophilus paragallinarum is a causative organism responsible for infectious coryza of chickens. Infectious coryza is an acute upper respiratory tract disease of chickens, which is of worldwide economic significance and affects both broiler and layer flocks. The disease is manifested primarily by a decrease in egg production (10-40%; Thornton & Blackall, 1984, Aust. Vet. J. 61 251) in layer flocks and retardation of growth due to decreased feed and water consumption in breeder and broiler flocks. The most common clinical symptoms include nasal discharge, facial oedema, lacrimation, anorexia and diarrhoea (Blackall, 1989, Clin. Microbiol. Rev. 2 270).

At present, inactivated whole cell vaccines against H. paragallinarum are available and are considered relatively effective (Blackall, 1989, supra). However, killed whole cell vaccines have limitations. The major problem with the current whole cell inactivated vaccines is that they do not provide cross serovar protection, i.e. they only protect against the serovar(s) present in the vaccine. Another limitation of whole cell inactivated vaccines is that since only limited serovar protection is afforded by those serovars in the vaccine, the introduction of new strains/serovars into a particular locality can produce antigenic pressure on the vaccine (Yamamoto, 1984, In: Diseases of Poultry, 8th Ed. Hofstad et al., Eds pp 178-186). This can result in uncontrolled infection of infectious coryza in that particular locality. Therefore, the use of local strains in vaccines is highly recommended (Bragg et al., 1996, Onderspoort J. Vet. Res. 63 217).

The serotyping antigen is the surface expressed haemagglutinin (HA). The most widely recognised serotyping scheme for H. paragallinarum is that of Page (Page, 1962, Am. J. Vet. Res. 23 85) and defines three serovars (A, B and C) based on agglutination activity. The Kume scheme (Kume et al., 1983, J. Clin. Microbiol. 17 1958) is related to the Page system but is based on haemagglutination inhibition activity. The Kume scheme groups H. paragallinarum into three serogroups which are further sub-divided into nine serovars. It has been established that the three serogroups recognised by the Kume scheme correspond to the three serovars of the Page system (Blackall et al., 1990, J. Clin. Microbiol. 28 1185). Although the Page system is the most widely used worldwide for serotyping of H. paragallinarum, it lacks the ability to type many strains. A significant number of isolates can not be serotyped by the Page agglutination system due to nonagglutination or autoagglutination (Thornton & Blackall, 1984, supra; Blackall & Eaves, 1988, Aust. Vet. J. 65 362; Eaves et al., 1989, J. Clin. Microbiol. 27 1510). Due to the technical difficulties and demand for a high level of expertise required in the assignment of serovars, the Kume system is not as widely applied as the Page scheme. Consequently, there is still a real need for alternate serotyping methodologies for H. paragallinarum.

In addition to use in serotyping, the haemagglutinin antigen (HA) plays a significant role in pathogenicity and immunogenicity of H. paragallinarum and previous reports have reported that it could form the basis of a novel vaccine against infectious coryza (Takagi et al., 1991a, J. Vet Med. Sci. 53 917.). The HA of H. paragallinarum is known to be serovar specific (Iritani et al., 1981, Avian Dis. 25 479; Kume et al., 1980, Am. J. Vet. Res. 41 97) and is considered to be a protective antigen because of the correlation between titre of haemagglutination inhibition (HI) antibody and protective immunity mediated in chickens (Iritani et al., 1981, supra; Kume et al., 1980, supra). The mechanism of protection, however, is not yet clearly understood.

The first report of isolation of a HA protein in substantially pure form (Iritani et al., 1980, Am. J. Vet. Res. 41 2114; U.S. Pat. No. 4,247,539) did not provide a corresponding protein or DNA sequence.

Subsequently, Takagi et al., 1991a, supra cloned a gene encoding a haemagglutination activity and claimed to have expressed a recombinant haemagglutinin of Haemophilus paragallinarum serotype A in Escherichia coli. The protein was expressed in E. coli and immunoblots using a monoclonal antibody directed against the haemagglutinin were positive. Neither the protein sequence nor an encoding DNA sequence were described, nor were the size of the gene or relative molecular weight of protein. Immunization of chickens with E. coli expressing this protein protected a minority of chickens from challenge with the same strain of H. paragallinarum from which the gene was cloned. There have been no further published developments which confirm, or otherwise, the identity of this putative haemagglutinin gene or its encoded product.

However, reference is made to European Patent 870828 which describes a polypeptide of around 130 kDa which induces the production of haemagglutination-inhibiting antibodies and protects against infectious coryza caused by H. paragallinarum serotype A.

Reference is also made to U.S. Pat. No. 5,240,705 directed to a vaccine in the form of a membrane fraction of H. paragallinarum cells comprising a 38 kDa outer-membrane protein. Again, neither the DNA sequence encoding this 38 kDa protein nor the amino acid sequence of the protein are disclosed in U.S. Pat. No. 5,240,705.

### SUMMARY OF THE INVENTION

In a first aspect, the present invention provides a recombinant haemagglutinin polypeptide of Haemophilus paragallinarum.

In a second aspect, the invention provides an isolated polypeptide comprising an amino acid sequence defined by residues 41-50 of any one of SEQ ID NOS:1-12.

In one embodiment, the isolated polypeptide has an amino acid sequence selected from the group consisting of the amino acid sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

In a preferred embodiment, the isolated polypeptide is a mature polypeptide having an N-terminus defined by the amino acid sequence APQANTFYAGAKAG (SEQ ID NO:13).

It will therefore be understood that the aforementioned polypeptides are examples of “HagA polypeptides of the invention”.

Preferably, the polypeptides of the invention when administered to an avian, are capable of eliciting an immune response.

Preferably, said immune response provides protection against one or more strains of Haemophilus paragallinarum.

In a third aspect, there is provided an isolated nucleic acid encoding a polypeptide according to the first- and second-mentioned aspects.

Preferably, the isolated nucleic acid encodes a mature HagA polypeptide and, more preferably, comprises the sequence of nucleotides: 5′-GCACCACAAGCAAAYACTTTCTATGCTGGTGCAAAAGCGGGC-3′ wherein Y=pyrimidine (SEQ ID NO:14).

Preferably, the isolated nucleic acid comprises a sequence of nucleotides defined by nucleotides 121-150 of any one of SEQ ID NOS:15-25.

Even more preferably, the isolated nucleic acid comprises a nucleotide sequence selected from the group consisting of the nucleotide sequences set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24 and SEQ ID NO:25.

The aforementioned isolated nucleic acids are examples of “hagA nucleic acids of the invention”.

Also contemplated are homologs, fragments, variants and derivatives of HagA polypeptides and isolated hagA nucleic acids of the invention.

In a fourth aspect, the invention resides in an expression construct comprising an isolated nucleic acid according to the third-mentioned aspect, wherein said sequence is operably linked to one or more regulatory nucleic acids in an expression vector.

In a fifth aspect, the invention provides a host cell comprising an expression construct according to the fourth-mentioned aspect.

Preferably, the host cell is a bacterium.

In a preferred embodiment, the host cell is an attenuated Salmonella or Mycoplasma bacterium.

In a sixth aspect of the invention, there is provided a method of producing a recombinant HagA polypeptide according to the first- or second-mentioned aspects, said method comprising the steps of:

(i) culturing a host cell containing an expression vector according to the fourth-mentioned aspect such that said recombinant polypeptide is expressed in said host cell; and

(ii) isolating said recombinant polypeptide.

In a seventh aspect, the invention provides an antibody or antibody fragment that binds to a HagA polypeptide of the invention, fragment, variant or derivative thereof.

In an eighth aspect, the invention provides a method of detecting H. paragallinarum including the step of detecting the presence of an isolated HagA polypeptide fragment or derivative which indicates the presence of H. paragallinarum.

Preferably, detection is performed using an antibody capable of binding said isolated HagA polypeptide fragment or derivative.

In a ninth aspect, the invention provides a method of detecting H. paragallinarum including the step of using an isolated HagA polypeptide, fragment or derivative of the invention to detect an H. paragallinarum-specific antibody in a sample obtained from an avian, wherein the presence of said antibody is indicative of said infection.

In a tenth aspect, the invention provides a method of detecting H. paragallinarum including the step of detecting an isolated nucleic acid according to the third-mentioned aspect, which isolated nucleic acid indicates the presence of H. paragallinarum.

In one embodiment of this aspect, the invention provides serovar- or strain-specific detection of H. paragallinarum bacteria, preferably using PCR.

In an eleventh aspect, the invention extends to the use of the polypeptide according to the first- and second-mentioned aspect, the use of the nucleic acids according to the third-mentioned aspect or the use of the antibody or antibody fragments mentioned above in a kit for detecting H. paragallinarum bacteria in a biological sample.

It will be appreciated that the detection methods according to the aforementioned aspects are preferably directed to detection of H. paragallinarum bacteria, or a bacterially-derived HagA polypeptide and/or hagA nucleic acid, in a biological sample obtained from an avian.

In a twelfth aspect of the invention, there is provided a pharmaceutical composition comprising an isolated polypeptide according to the first- or second-mentioned aspect.

Preferably, said pharmaceutical composition is a vaccine.

In a thirteenth aspect, the invention provides a method of immunizing an avian against H. paragallinarum infection, said method including the step of administering the above-mentioned vaccine to said avian.

Preferably, the avian according to the aforementioned aspects is a chicken.

In a fourteenth aspect, the invention provides a method of identifying an immunogenic fragment of a HagA polypeptide, variant or derivatives according to the first- or second-mentioned aspect, comprising the steps of:

(i) producing a fragment of said polypeptide, variant or derivative;

(ii) administering said fragment to a mammal or avian; and

(iii) detecting an immune response in said mammal or avian, which response includes production of elements which specifically bind H. paragallinarum and/or said polypeptide, variant or derivative, and/or a protective effect against H. paragallinarum infection.

Preferably, the mammal is a mouse or rabbit.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES AND TABLES

TABLE 1: Examples of conservative amino acid substitutions.

TABLE 2: H. paragallinarum strains used in the sequencing of the hagA gene. (P) indicates the reference strain for the Page serotyping scheme; (K) indicates the reference strain for the Kume serotyping scheme. Primer sets required for amplification of the hagA gene are indicated in the far right column. Primer set (1) consists of primers HA8/HA10; primer set (2) consists of primers HA8/HA11.

TABLE 3: Nucleotide sequences of oligonucleotide primers (SEQ ID NOS:26-36) used in inverse PCR, sequencing and cloning of the hagA gene of serovars A, B and C of H. paragallinarum. The core region of the hagA gene was amplified using HA1 and HA2. Primers HA3, HA5, HA6 and HA7 were used in inverse PCR to amplify upstream and downstream regions flanking the core region and sequencing of the gene. HA8 and HA11 primers anneal to the intergenic regions of the HA to amplify the full-length gene (HA8 upstream, HA11 downstream). HA12 and HA13 primers were used for cloning. The GCA (bold) in HA12 encodes the first amino acid in the mature form of the protein after processing of the leader sequence. The TAA (bold) in HA13 encodes the stop codon.

FIG. 1: Partial purification of H. paragallinarum strain 0083 whole cells, using ammonium sulphate [(NH₄)₂SO₄] precipitation revealed the presence of a 39 kDa protein in all three precipitation concentrations. The 39 kDa protein was recognized by the anti-haemagglutinin monoclonal antibody (MAb4D). The 0-20% fraction contained the highest concentration of the 39 kDa protein and was subsequently used for N-terminal sequencing. (A) SDS-PAGE gel stained with Coomassie brilliant blue. (B) Anti-haemagglutinin monoclonal antibody. (MAb4D) immunoblot. Lane 1, molecular mass marker (Benchmark, GibcoBRL); lane 2, H. paragallinarum strain 0083 whole cell extract; lane 3, 0-20% ammonium sulfate fraction; lane 4, 20-40% fraction; lane 5, 40+% fraction. The molecular mass (in kilodaltons) is shown on the left.

FIG. 2: Schematic representation of inverse PCR products and primers used to identify the full length sequence of the H. paragallinarum hagA gene. The shaded arrow represents the ORF (hagA) and the direction of transcription. The core region represents the sequence obtained from HA1/HA2 amplification. Hind5/6 represents the inverse PCR product obtained from HindIII restriction digest of strain Modesto chromosomal DNA. Primers HA5 and HA6 were used to amplify and sequence the Hind5/6 fragment. Bfa3/7 represents the inverse PCR product obtained from BfaI restriction digest of strain Modesto chromosomal DNA. Primers HA3 and HA7 were used to amplify and sequence the Bfa3/7 fragment.

FIG. 3: Alignment of the deduced amino acid sequence of H. paragallinarum strain HP14 hagA (SEQ ID NO:5) with the P5 protein of H. influenzae (Genbank accession number L20309; SEQ ID NO:37) The sequences were aligned using the Multalin program, version 5.3.3 (Corpet, 1988, Nucleic Acids Res. 16 10881) to produce a consensus sequence (SEQ ID NO:38). Consensus symbols are: !=I or V; $=L or M; %=F or Y and #=N, D, E, B or Z, wherein B=asp or asn and Z=glu or gln.

FIG. 4: Alignment of the deduced amino acid sequences of the 11 H. paragallinarum strains (hagA gene). The amino acid sequences of H. paragallinarum strains 0083 (serovar A; SEQ ID NO:1), 221 (serovar A; SEQ ID NO:2), 2403 (serovar A; SEQ ID NO: 3), E-3C (serovar A; SEQ ID NO:4), HP14 (serovar A; SEQ ID NO:5), 0222 (serovar B; SEQ ID NO:6), 2671 (serovar B; SEQ ID NO:7), Modesto (serovar C; SEQ ID NO:8), H-18 (serovar C; SEQ ID NO:9), SA-3 (serovar C; SEQ ID NO:10) and HP60 (serovar C; SEQ ID NO:11) were aligned using the Multalin program, version 5.3.3 (Corpet, 1988, supra). Consensus symbols for the consensus amino acid sequence (SEQ ID NO:12) are: !=I or V; $=L or M; %=F or Y and #=N, D, E, B or Z, wherein B=asp or asn and Z=glu or gln. The predicted signal peptide cleavage site is indicated by an arrow. The N-terminal sequence of the mature form of the protein (SEQ ID NO:13) is underlined. Non-conserved amino acids are indicated by bolding or by dashes. Regions between bolded residues or dashes are defined as conserved regions. Conserved regions absolutely unique to all HagA polypeptides are defined by residues 41-50 and 131-140 of each of the sequences shown in FIG. 4.

FIG. 5: Alignment of the nucleotide sequences of the 11 H. paragallinarum strains (hagA gene). The nucleotide sequences of H. paragallinarum strains 0083 (serovar A; SEQ ID NO:15), 221 (serovar A; SEQ ID NO:16), 2403 (serovar A; SEQ ID NO:17), E-3C (serovar A; SEQ ID NO:18), HP14 (serovar A; SEQ ID NO;19), 0222 (serovar B; SEQ ID NO:20), 2671 (serovar B; SEQ ID NO:21), Modesto (serovar C; SEQ ID NO:22), H-18 (serovar C; SEQ ID NO:23), SA-3 (serovar C; SEQ ID NO:24) and HP60 (serovar C; SEQ ID NO:25) were aligned using the Multalin program, version 5.3.3 (Corpet, 1988, supra). The predicted start of the coding region for the mature protein is indicated by an arrow. The 5′ sequence encoding the N-terminal amino acid sequence (SEQ ID NO:13) obtained from strain 0083 is underlined (the consensus 5′ sequence is SEQ ID NO:14). The double underline indicates the codon encoding the last amino acid of the signal sequence. Non-conserved nucleotides are indicated by bolding or by dashes. Regions between bolded residues or dashes are defined as conserved regions. Conserved regions absolutely unique to all hagA nucleic acids are defined by residues 121-150 of each of the sequences shown in FIG. 5. Residues 391-420 are also unique and conserved except for a synonymous C/T variation at residue 417.

FIG. 6: Phylogenetic tree representing the relationship between the full length hagA gene sequences of the 11 serotyping reference strains of H. paragallinarum. Strains 0083, 221, 2403, E-3C, and HP14 belong to Page serovar A; strains 0222 and 2671 belong to Page serovar B and strains Modesto, H-18, SA-3 and HP60 belong to Page serovar C. The evolutionary distance tree was constructed using the PAM-Dayhoff model for amino acids and Neighbor Joining (ARB software package).

FIG. 7: Purification of recombinant His-tagged HagA protein. (A) Coomassie blue stained SDS-PAGE gel of purified protein. Lane 1; molecular mass ladder (Benchmark, GibcoBRL), lane 2; M15/pQE30hagA induced cell pellet, lane 3; purified recombinant His-tagged HagA protein. (B) Immunoblot using anti-haemagglutinin monoclonal antibody (MAb4D). Lane 4; molecular mass ladder (Benchmark, GibcoBRL), lane 5; H. paragallinarum strain HP14 whole cell preparation, lane 6; purified recombinant HagA protein (1 μg).

FIG. 8: ELISA analysis of antibody production by chickens immunized with HagA protein. Titre is expressed as the reciprocal of the last dilution which showed reactivity.

### DETAILED DESCRIPTION OF THE INVENTION

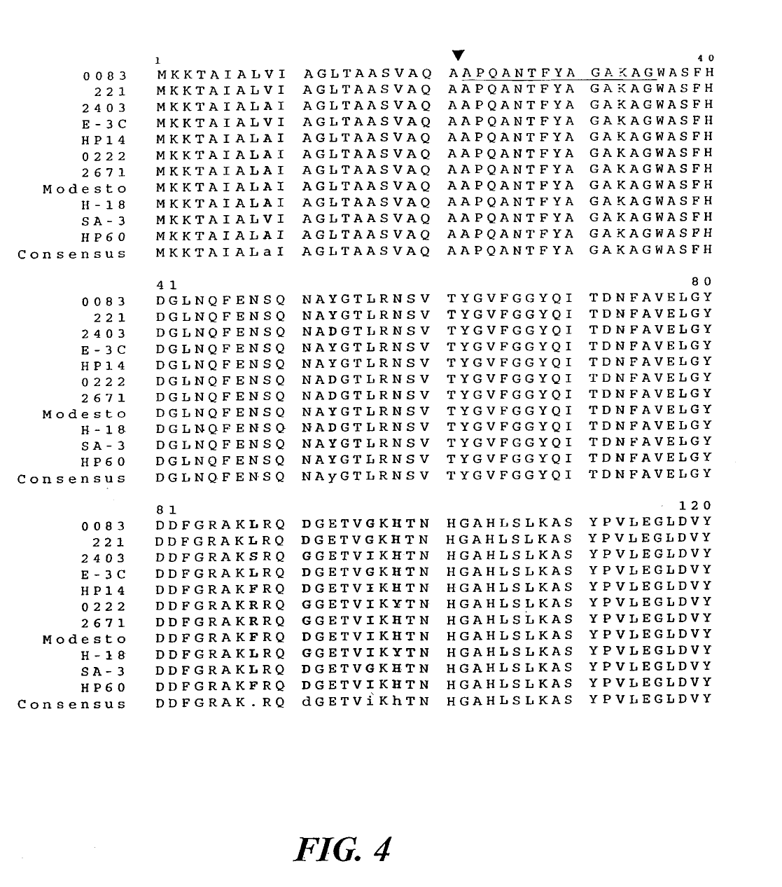

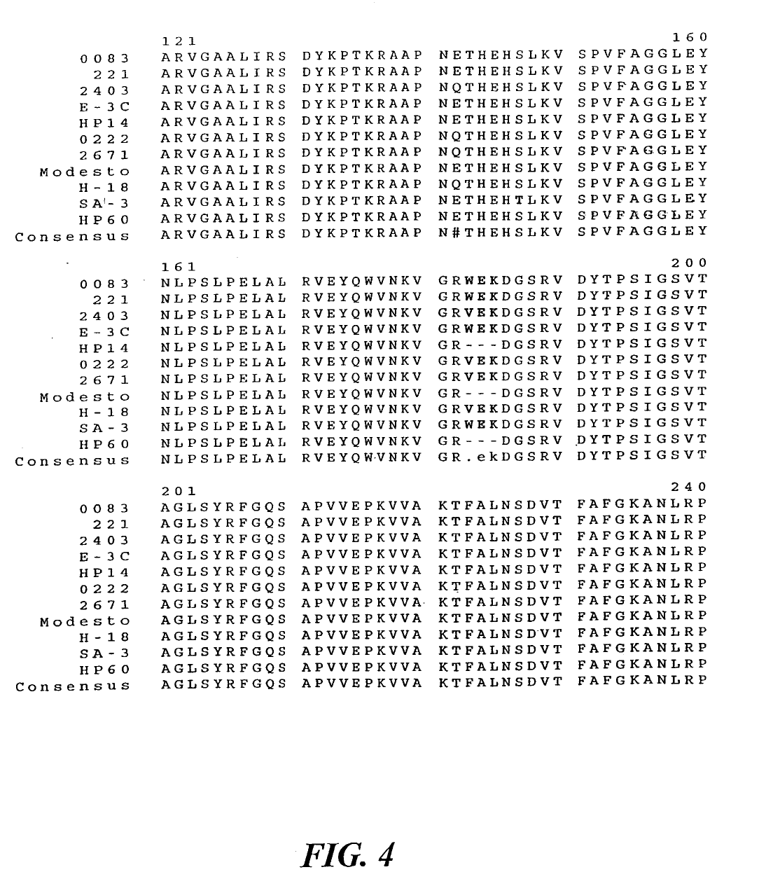

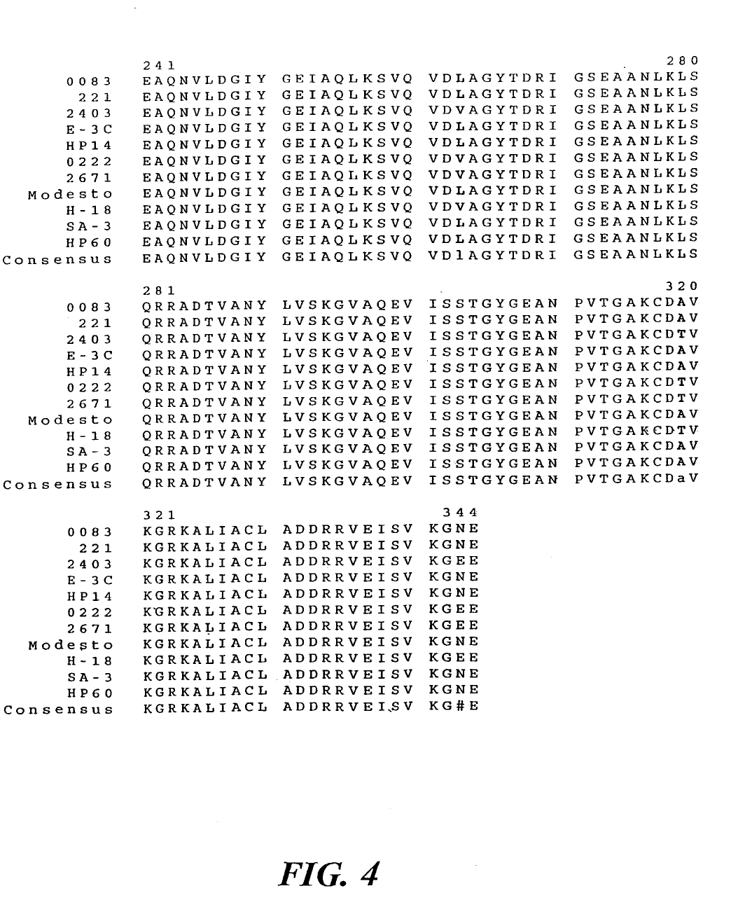

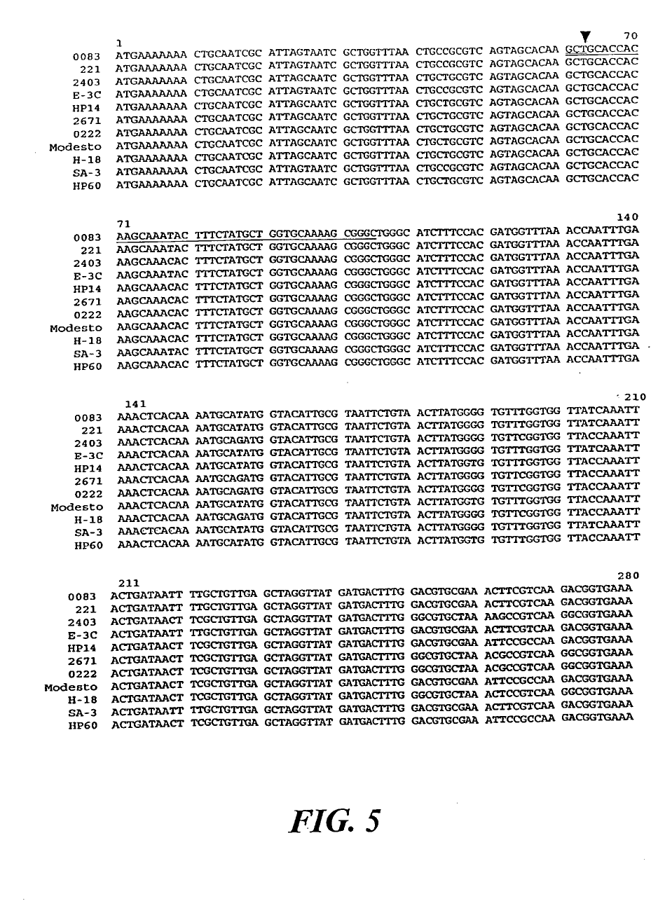

The present invention is predicated, at least in part, on the isolation of Haemophilus paragallinarum haemagglutinin polypeptides and encoding nucleic acids by the present inventors. The HagA polypeptides set forth in FIG. 4 and SEQ ID NOS:1-11 correspond to HagA polypeptides from eleven (11) distinct serovars of Haemophilus paragallinarum, that appear to constitute new members of the P5 group of bacterial proteins. To date, the amino acid sequences and encoding nucleotide sequences of Haemophilus paragallinarum HagA polypeptides have remained elusive. The present inventors therefore have provided isolated HagA polypeptides and nucleic acids which will revolutionize Haemophilus paragallinarum detection and serotyping, and facilitate the large-scale production of recombinant HagA vaccines for mass immunization against infectious coryza in chickens.

The term “recombinant” as used herein means artificially produced through human manipulation of genetic material, such as involving techniques generally falling within the scope of “recombinant DNA technology” as is well understood in the art.

By “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in recombinant or native form.

Isolated HagA Polypeptides

The isolated polypeptides set forth in SEQ ID NO: 1; SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11 represent immature, unprocessed forms of the HagA polypeptides of the invention.

As is clear from FIG. 4, an N-terminal signal peptide is cleaved to produce a mature, processed HagA polypeptide wherein APQANTFYAGAKAG (SEQ ID NO:13) defines the N-terminus of the mature polypeptide.

Residues 41-50 and 131-140 of SEQ ID NOS:1-11 are unique to, and absolutely conserved between, each of the HagA polypeptides.

SEQ ID NO:12 is a consensus sequence based on the eleven HagA sequences set forth in SEQ ID NOS:1-11, and serves to exemplify the high degree of sequence conservation between HagA polypeptides of the invention.

By “polypeptide” is also meant “protein”, either term referring to an amino acid polymer which may include natural and/or non-natural amino acids as are well known in the art.

A “peptide” is a protein having no more than fifty (50) amino acids.

A peptide is an example of a polypeptide “fragment”.

In one embodiment, a “fragment” includes an amino acid sequence which constitutes less than 100%, but at least 20%, preferably at least 50%, more preferably at least 80% or even more preferably at least 90% of said polypeptide.

In another embodiment, a “fragment” is a small peptide, for example of at least 6, preferably at least 10 and more preferably at least 20 amino acids in length, which comprises one or more antigenic determinants or epitopes. Larger fragments comprising more than one peptide are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcal V8-protease. The digested fragments can be purified by, for example, high performance liquid chromatographic (HPLC) techniques.

Such fragments may also include “biologically active fragments” that have at least 1%, preferably at least 5% or more preferably at least 25% of a biological activity of a HagA polypeptide of the invention.

In this context, biological activity may include immunogenicity, antigenicity, or haemagglutinin activity.

The mature HagA polypeptides of the invention are examples of biologically active fragments of the polypeptides set forth in SEQ ID NO:1; SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

As used herein, “variant” polypeptides are HagA polypeptides of the invention in which one or more amino acids have been replaced by different amino acids. It is well understood in the art that some amino acids may be changed to others with broadly similar properties without changing the nature of the activity of the polypeptide (conservative substitutions). Exemplary conservative substitutions in the polypeptide may be made according to TABLE 1.

Substantial changes in function are made by selecting substitutions that are less conservative than those shown in TABLE 1. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser)or no side chain (e.g., Gly).

The term “variant” also includes HagA polypeptides produced from allelic variants of the sequences exemplified in this specification.

Polypeptide variants fall within the scope of the term “polypeptide homologs”.

In an embodiment, polypeptide homologs of the invention share at least 63%, preferably at least 80% and more preferably at least 90% sequence identity with the amino acid sequences set forth in FIG. 4.

As generally used herein, a “homolog” shares a definable nucleotide or amino acid sequence relationship with a nucleic acid or polypeptide of the invention as the case may be.

Included within the scope of homologs are “orthologs”, which are functionally-related polypeptides and their encoding nucleic acids, isolated from organisms other than Haemophilus paragallinarum, such as other Haemophilus species.

Terms used herein to describe sequence relationships between respective nucleic acids and polypeptides include “comparison window”, “sequence identity”, “percentage of sequence identity” and “substantial identity”. Because respective nucleic acids/polypeptides may each comprise (1) only one or more portions of a complete nucleic acid/polypeptide sequence that are shared by the nucleic acids/polypeptides, and (2) one or more portions which are divergent between the nucleic acids/polypeptides, sequence comparisons are typically performed by comparing sequences over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference.

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

Thus, it is well within the capabilities of the skilled person to prepare polypeptide homologs of the invention, such as variants as hereinbefore defined, by recombinant DNA technology. For example, nucleic acids of the invention can be mutated using either random mutagenesis for example using transposon mutagenesis, or site-directed mutagenesis. The resultant DNA fragments are then cloned into suitable expression hosts such as E. coli using conventional technology and clones that retain the desired activity are detected. Where the clones have been derived using random mutagenesis techniques, positive clones would have to be sequenced in order to detect the mutation.

As used herein, “derivative” polypeptides are polypeptides of the invention which have been altered, for example by conjugation or complexing with other chemical moieties or by post-translational modification techniques as would be understood in the art. Such derivatives include amino acid deletions and/or additions to polypeptides of the invention, or variants thereof, wherein said derivatives elicit an immune response.

“Additions” of amino acids may include fusion of the polypeptides or variants thereof with other polypeptides or proteins. Particular examples of such proteins include Protein A, glutathione S-transferase (GST), maltose-binding protein (MBP), hexahistidine (HIS₆) and epitope tags such as FLAG and c-myc tags.

A preferred addition is a hexahistidine tag, which facilitates recombinant HagA polypeptide purification, as will be described in detail hereinafter.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

The invention also contemplates covalently modifying a HagA polypeptide, fragment or variant of the invention with dinitrophenol, in order to render it immunogenic in chickens

HagA polypeptides of the invention (inclusive of fragments, variants, derivatives and homologs in general) may be prepared by any suitable procedure known to those of skill in the art.

For example, a recombinant HagA polypeptide may be prepared by a procedure including the steps of:

(i) preparing an expression construct which comprises a hagA nucleic acid of the invention, operably linked to one or more regulatory nucleotide sequences;

(ii) transfecting or transforming a suitable host cell with the expression construct; and

(iii) expressing the recombinant polypeptide in said host cell.

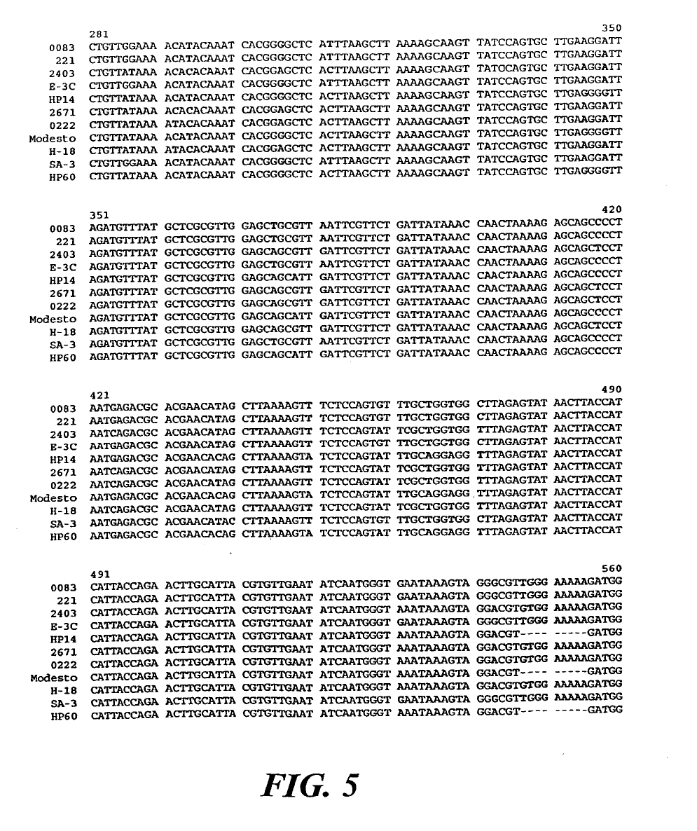

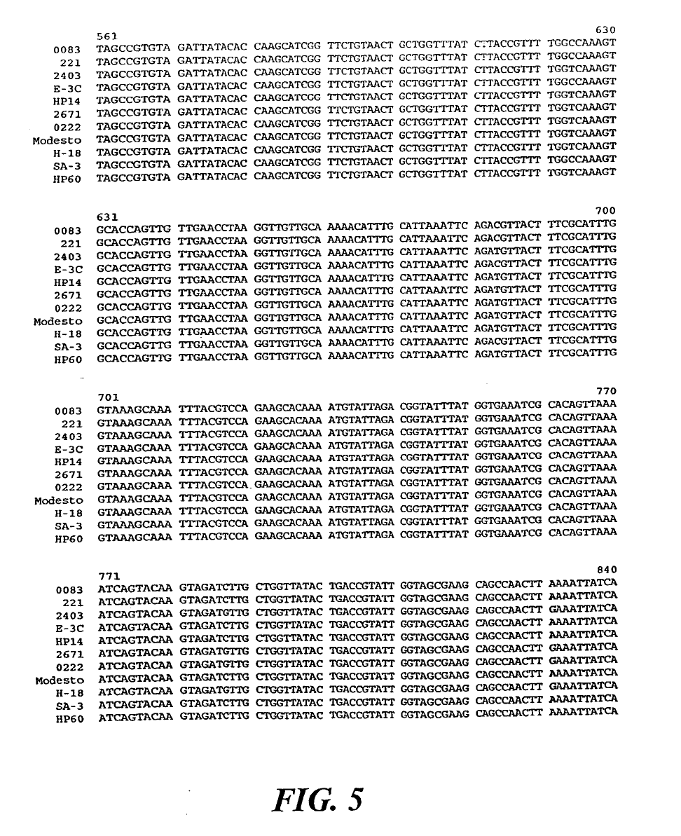

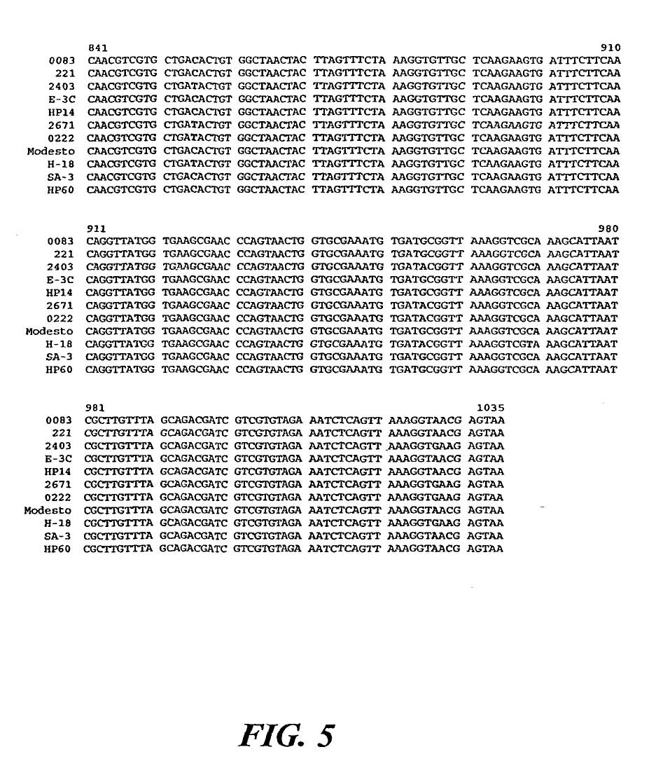

Preferably, the hagA nucleic acid at step (i) has a nucleotide sequence selected from the group consisting of the nucleotide sequences set forth in FIG. 5 (SEQ ID NOS: 15-25), or fragments of these that encode mature HagA polypeptides.

It will also be appreciated that the abovementioned method is suitable for producing recombinant polypeptides from nucleic acid homologs, as will be described in more detail hereinafter.

Suitable host cells for expression may be prokaryotic or eukaryotic.

Preferably, the host cell is prokaryotic.

Preferably, the prokaryotic cell is a bacterium.

Preferred bacteria are E. coli, or bacteria of the genus Salmonella and the genus Mycoplasma.

Alternatively, the host cell may be a eukaryotic cell, for example yeast, COS, Chinese Hamster Ovary (CHO) or SF9 cells that may be utilized with a baculovirus expression system.

For the purposes of host cell expression, the hagA nucleic acid is operably linked to one or more regulatory sequences in an expression vector.

An “expression vector” may be either a self-replicating extra-chromosomal vector such as a plasmid, or a vector that integrates into a host genome.

By “operably linked” is meant that said regulatory nucleotide sequence(s) is/are positioned relative to the hagAnucleic acid of the invention or homolog thereof, to initiate, regulate or otherwise control transcription of the nucleic acid.

Regulatory nucleotide sequences will generally be appropriate for the host cell used for expression, as regulatory sequences and host cell are often interdependent. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selectable marker genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide.

In order to express said fusion polypeptide, it is necessary to ligate the hagA nucleic acid or homolog into the expression vector so that the translational reading frames of the fusion partner and the operably linked nucleic acid coincide.

Well known examples of fusion partners include, but are not limited to, glutathione-S-transferase (GST), Fc portion of IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localization of the fusion polypeptide of the invention, or for isolating cells which express the fusion polypeptide of the invention. Flow cytometric methods such as fluorescence activated cell sorting (FACS) are particularly useful in this latter application.

Fusion partners may have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

Fusion partners according to the invention also include within their scope “epitope tags”, which are usually short peptide sequences for which a specific antibody is available. Well known examples of epitope tags for which specific monoclonal antibodies are readily available include c-myc, influenza virus haemagglutinin and FLAG tags.

As hereinbefore described, HagA polypeptides of the invention may be produced by culturing a host cell transformed with the aforementioned expression construct. The conditions appropriate for protein expression will vary with the choice of expression vector and the host cell. For example, the induction system used for protein system varies from one vector to another. This is easily ascertained by one skilled in the art through routine experimentation and reference to the appropriate product literature.

The recombinant protein may be conveniently prepared by a person skilled in the art using standard protocols as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6 and 7.

Isolated Nucleic Acids

The invention provides an isolated nucleic acid that encodes a HagA polypeptide of the invention.

In a preferred embodiment, the isolated nucleic acid comprises residues 121-150 of any one of SEQ ID NOS:15-25.

The isolated nucleic acids set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24 and SEQ ID NO:25 encode immature, unprocessed forms of HagA polypeptides.

Preferably, isolated hagA nucleic acids encode mature HagA polypeptides as hereinbefore defined.

The term “nucleic acid” as used herein designates single- or double-stranded mRNA, RNA, cRNA and DNA, said DNA inclusive of cDNA and genomic DNA.

A “polynucleotide” is a nucleic acid having eighty (80) or more contiguous nucleotides, while an “oligonucleotide” has eight (8) to eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The present invention also contemplates homologs of hagA nucleic acids of the invention.

In one embodiment, nucleic acid homologs encode polypeptide homologs of the invention, inclusive of variants, fragments and derivatives thereof.

In another embodiment, nucleic acid homologs share at least 60%, preferably at least 70%, more preferably at least 80%, and even more preferably at least 90% sequence identity with the nucleotide sequences of FIG. 5.

In yet another embodiment, nucleic acid homologs hybridize to the nucleotide sequences of FIG. 5 under at least low stringency conditions, preferably under at least medium stringency conditions and more preferably under high stringency conditions.

“Hybridize and Hybridization” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:

(i) A and T; and

(ii) C and G.

In RNA, complementary bases are:

(i) A and U; and

(ii) C and G.

In RNA-DNA hybrids, complementary bases are:

(i) A and U;

(ii) A and T; and

(iii) G and C.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acids having a high frequency of complementary bases will hybridize.

Reference herein to low stringency conditions includes and encompasses:

(i) from at least about 1% v/v to at least about 15% v/v formamide and from at least about 1 M to at least about 2 M salt for hybridisation at 42° C., and at least about 1 M to at least about 2 M salt for washing at 42° C.; and

(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:

(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and

(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

In general, washing is carried out at Tₘ=69.3+0.41 (G+C) %=−12° C. However, the Tₘ of a duplex DNA decreases by 1° C. with every increase of 1% in the number of mismatched bases.

Notwithstanding the above, stringent conditions are well known in the art, such as described in Chapters 2.9 and 2.10 of. Ausubel et al., supra, which are herein incorporated be reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20.

According to such methods, Southern blotting involves separating DNA molecules according to size by gel electrophoresis, transferring the size-separated DNA to a synthetic membrane, and hybridizing the membrane bound DNA to a complementary nucleotide sequence.

In dot blotting and slot blotting, DNA samples are directly applied to a synthetic membrane prior to hybridization as above.

An alternative blotting step is used when identifying complementary nucleic acids in a cDNA or genomic DNA library, such as through the process of plaque or colony hybridization. Other typical examples of this procedure is described in Chapters 8-12 of Sambrook et al., supra which are herein incorporated by reference.

Typically, the following general procedure can be used to determine hybridization conditions. Nucleic acids are blotted/transferred to a synthetic membrane, as described above. A wild type nucleotide sequence of the invention is labeled as described above, and the ability of this labeled nucleic acid to hybridize with an immobilized nucleotide sequence analyzed.

A skilled addressee will recognize that a number of factors influence hybridization and detection of hybridized nucleic acids. The specific activity of radioactively labeled polynucleotide sequence should typically be greater than or equal to about 10⁸ dpm/μg to provide a detectable signal. A radiolabeled nucleotide sequence of specific activity 10⁸ to 10⁹ dpm/μg can detect approximately 0.5 pg of DNA. It is well known in the art that sufficient DNA must be immobilized on the membrane to permit detection. It is desirable to have excess immobilized DNA, usually 10 μg. Adding an inert polymer such as 10% (w/v) dextran sulfate (MW 500,000) or polyethylene glycol 6000 during hybridization can also increase the sensitivity of hybridization (see Ausubel et al., supra at 2.10.10).

To achieve meaningful results from hybridization between a nucleic acid immobilized on a membrane and a labeled nucleic acid, a sufficient amount of the labeled nucleic acid must be hybridized to the immobilized nucleic acid following washing. Washing ensures that the labeled nucleic acid is hybridized only to the immobilized nucleic acid with a desired degree of complementarity to the labeled nucleic acid.

Methods for detecting labeled nucleic acids hybridized to an immobilized nucleic acid are well known to practitioners in the art. Such methods include autoradiography, chemiluminescent, fluorescent and colorimetric detection.

In an embodiment, nucleic acid homologs of the invention may be prepared according to the following procedure:

(i) obtaining a nucleic acid extract from a bacterium;

(ii) using one or more primers derived from a hagA nucleic acid of the invention to amplify, via nucleic acid amplification techniques, one or more amplification products from said nucleic acid extract.

Preferably, the bacterium is of the genus Haemophilus such as Haemophilus influenzae or Haemophilus paragallinarum.

More preferably, the bacterium is of the species Haemophilus paragallinarum.

Suitably, the primers may be degenerate or non-degenerate primers derived from a hagA nucleic acid of the invention.

In this context, “derived from” means that the primer(s) include nucleotide sequence from a hagA nucleic acid of the invention and, optionally, other nucleotides that allow sufficient degeneracy to anneal or hybridize to related but non-identical homologous nucleic acids.

Examples of non-degenerate primers potentially suitable for isolation and detection of homologous nucleic acids include SEQ ID NOS:26-36 as shown in Table 2.

Suitable nucleic acid amplification techniques are well known to the skilled addressee, and include polymerase chain reaction (PCR) as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International Publication WO 92/01813) and Lizardi et al., (International Publication WO 97/19193) which are incorporated herein by reference; nucleic acid sequence-based amplification (NASBA) as for example described by Sooknanan et al., 1994, Biotechniques 17 1077) which is incorporated herein by reference; ligase chain reaction (LCR) as for example described in International Publication WO 89/09385 which is incorporated by reference herein; and Q-β replicase amplification as for example described by Tyagi et al., 1996, Proc. Natl. Acad. Sci. USA 93 5395, which is incorporated herein by reference.

The preferred nucleic acid sequence amplification technique is PCR, as will be described in detail hereinafter.

As used herein, an “amplification product” refers to a nucleic acid product generated by nucleic acid amplification techniques.

Antibodies

The invention also contemplates antibodies against the HagA polypeptides, fragments, variants and derivatives of the invention. Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative of the invention. For example, the antibodies may comprise polyclonal antibodies. Such antibodies may be prepared for example by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al, CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies which comprise Fc or Fab fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No. 5,091,513, European Patent No. 239400 or the article by Winter & Milstein, 1991, Nature 349 293, which are incorporated herein by reference.

The antibodies of the invention may be used for affinity chromatography in isolating native or recombinant HagA polypeptides. For example reference may be made to immunoaffinity chromatographic procedures described in Chapter 9.5 of Coligan et al., CURRENT PROTOCOLS IN IMMUNOLOGY, supra.

For example, the anti-HagA antibodies may be used for serological analysis such as by ELISA.

However, it will be appreciated that any suitable technique for determining formation of antibody complex may be used. For example, an antibody or antibody fragment according to the invention having a label associated therewith may be utilized in immunoassays. Such immunoassays may include, but are not limited to, radioimmunoassays (RIAs), enzyme-linked immunosorbent assays (ELISAs) and immunochromatographic techniques (ICTs) which are well known to those of skill in the art.

For example, reference may be made to Chapter 7 of Coligan et al., CURRENT PROTOCOLS IN IMMUNOLOGY, supra which discloses a variety of immunoassays that may be used in accordance with the present invention. Immunoassays may include competitive assays as understood in the art.

The label associated with the antibody or antibody fragment may include the following:

(A) direct attachment of the label to the antibody or antibody fragment;

(B) indirect attachment of the label to the antibody or antibody fragment; i.e., attachment of the label to another assay reagent which subsequently binds to the antibody or antibody fragment; and

(C) attachment to a subsequent reaction product of the antibody or antibody fragment.

The label may be selected from a group including a chromogen, a catalyst, an enzyme, a fluorophore, a chemiluminescent molecule, a lanthanide ion such as Europium (Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as labels is disclosed in United States Patent Specifications U.S. Pat. Nos. 4,366,241, 4,843,000, and 4,849,338, each of which is herein incorporated by reference. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution.

Fluorophores may be selected from a group including fluorescein isothiocyanate (FITC), tetramethylrhodamine isothiocyanate (TRITC), allophycocyanin (APC), Texas Red (TR), Cy5 or R-Phycoerythrin (RPE). Examples of useful fluorophores may be found, for example, in U.S. Pat. Nos. 4,520,110 and 4,542,104 which are herein incorporated by reference.

Pharmaceutical Compositions A further feature of the invention is the use of the HagA polypeptides, fragments, variants or derivatives of the invention (“immunogenic agents”) as actives in a pharmaceutical composition. Suitably, the pharmaceutical composition comprises a pharmaceutically-acceptable carrier, diluent or excipient.

Preferably, said pharmaceutical composition comprises one or more mature HagA polypeptides.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a chicken with the composition of the invention. For example, oral, rectal, parenteral, sublingual, buccal, intravenous, intra-articular, intramuscular, intradermal, subcutaneous, inhalational, intraocular, intraperitoneal, intracerebroventricular, transdermal and the like may be employed. Intra-muscular and subcutaneous injection is appropriate, for example, for administration of immunogenic compositions, vaccines and DNA vaccines.

Preferred administration routes in chickens include intramuscular, intranasal, oral, in ovo, intraocular and subcutaneous.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

Vaccines and Methods of Immunization

The above compositions may be used as therapeutic or prophylactic vaccines for administration to an avian, preferably a chicken. Vaccines and methods of immunization may therefore be directed to prevention of infection by H. paragallinarum or treatment of existing H. paragallinarum infection.

Any suitable procedure is contemplated for producing and administering said vaccines. Exemplary procedures include, for example, those described in NEW GENERATION VACCINES (1997, Levine et al., Marcel Dekker, Inc. New York, Basel Hong Kong) which is incorporated herein by reference.

In this regard, reference is made to U.S. Pat. No. 5,770,213, Australian Patent 704882, Reid & Blackall, 1987, 31 59 and Webb & Cripps, 2000, Infect. Immun. 68 377 (which are each incorporated herein by reference) which describe immunization methods which may be applicable to immunogenic agents of the present invention.

An immunogenic agent according to the invention can be mixed, conjugated or fused with other antigens, including B or T cell epitopes of other antigens. In addition, it can be conjugated to a carrier as described below.

When an haptenic peptide of the invention is used (i.e., a peptide which reacts with cognate antibodies, but cannot itself elicit an immune response), it can be conjugated with an immunogenic carrier. Useful carriers are well known in the art and include for example: thyroglobulin; albumins such as human serum albumin; toxins, toxoids or any mutant crossreactive material (CRM) of the toxin from tetanus, diptheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a haptenic peptide of the invention can be coupled to a T cell epitope of a bacterial toxin, toxoid or CRM. In this regard, reference may be made to U.S. Pat. No. 5,785,973 which is incorporated herein by reference.

In addition, a HagA polypeptide, fragment, variant or derivative of the invention may act as a carrier protein in vaccine compositions directed against Haemophilus paragallinarum, or against other bacteria or viruses.

The immunogenic agents of the invention may be administered as multivalent subunit vaccines in combination with antigens of Haemophilus paragallinarum, or antigens of other organisms. Alternatively or additionally, they may be administered in concert with oligosaccharide or polysaccharide components of Haemophilus paragallinarum.

The vaccines can also contain a physiologically-acceptable carrier, diluent or excipient such as water, phosphate buffered saline and saline.

The vaccines and immunogenic compositions may include an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

The immunogenic agents of the invention may be expressed by attenuated viral and/or bacterial hosts. By “attenuated” is meant viruses or bacteria (for example transformed with an expression construct of the invention) that are either naturally, or have been rendered, substantially avirulent. A virus or bacterium may be rendered substantially avirulent by any suitable physical (e.g., heat treatment) or chemical means (e.g., formaldehyde treatment) or by genetic manipulation. By “substantially avirulent” is meant a virus or bacterium whose ability to cause disease has been destroyed. Ideally, the pathogenicity of the virus or bacterium is destroyed without affecting immunogenicity. From the foregoing, it will be appreciated that attenuated viral and bacterial hosts may comprise live or inactivated viruses and bacteria.

Attenuated viral and bacterial hosts which may be useful in a vaccine according to the invention may comprise viral vectors inclusive of Marek's disease virus, adenovirus and cytomegalovirus and attenuated Salmonella or Mycoplasma strains. Live vaccines are particularly advantageous because they lead to a prolonged stimulus that can confer substantially long-lasting immunity. For example, with regard to Salmonella or Mycoplasma strains, upon introduction of an attenuated bacterium harbouring an expression construct of the invention to a chicken, the HagA polypeptide or fragment expressed by the bacterium will suitably elicit a host immune response. In this regard, reference is particularly made to U.S. Pat. No. 6,001,348 for a description of such an approach using Mycoplasma synoviae.

Multivalent vaccines can be prepared from one or more microorganisms that express different epitopes of Haemophilus paragallinarum (e.g., other surface proteins or epitopes of Haemophilus paragallinarum). In addition, epitopes of other pathogenic microorganisms can be incorporated into the vaccine.

A wide variety of other vectors useful for therapeutic administration or immunization with the immunogenic agents of the invention will be apparent to those skilled in the art from the present disclosure.

In a further embodiment, the nucleotide sequence may be used as a vaccine in the form of a “naked DNA” vaccine as is known in the art. For example, an expression vector of the invention may be introduced into a chicken, where it causes production of a polypeptide in vivo, against which the host mounts an immune response as for example described in Barry et al., 1995, Nature 377 632 which is hereby incorporated herein by reference.

Detection Methods

The present invention also provides detection of Haemophilus paragallinarum in a biological sample.

Preferably, the biological sample is a nucleic acid sample obtained from an avian.

Preferably, the avian is a chicken.

Detection may utilize a kit comprising one or more of a HagA polypeptide, fragment, variant, derivative, antibody, antibody fragment or nucleic acid according to the invention. The kit may also optionally include appropriate reagents for detection of labels, positive and negative controls, washing solutions, dilution buffers and the like.

In another embodiment, a nucleic acid-based detection kit may include (i) a hagA nucleic acid according to the invention (which may be used as a positive control), (ii) one or more primers according to the invention, and optionally a DNA polymerase, DNA ligase etc depending on the nucleic acid amplification technique employed.

A preferred method of detection comprises the steps of:

(i) obtaining a nucleic acid sample from an avian;

(ii) using one or more primers derived from a hagA nucleic acid of the invention together with a nucleic acid sequence amplification technique (as hereinbefore defined) to produce one or more amplification products from the sample obtained in step (i); and

(iii) detecting the one or more amplification products produced at step (ii) and correlating the amplification products so detected with the presence or absence of a particular H. paragallinarum serovar or strain.

This preferred method facilitates detection of hagA nucleic acids of the invention, homologous nucleic acids and fragments thereof.

Typically, the amplification product(s) detected at step (iii) will correspond to a fragment of a hagA nucleic acid of the invention or said homologous nucleic acid.

Preferably, the nucleic acid sequence amplification technique is PCR.

Preferably, the nucleic acid sample is obtained from a chicken.

Preferred primers are SEQ ID NOS:26-36 as set forth in Table 3.

Alternatively, primers used for detection may be degenerate, as hereinbefore defined.

The present invention also contemplates serovar-specific PCR detection where sufficient nucleic acid sequence divergence exists between serovars. As will be appreciated by the skilled person, specific primers can be designed so as to allow differential amplification of nucleic acids and thereby facilitate serovar-specific nucleic acid amplification.

Preparation of Immunoreactive Fragments

The invention also extends to a method of identifying an immunoreactive fragment of a HagA polypeptide, variant or derivatives according to the invention. This method essentially comprises generating a fragment of the polypeptide, variant or derivative, administering the fragment to a chicken or mammal such as a mouse or rabbit; and detecting an immune response in the chicken. Such response will include production of elements which specifically bind Haemophilus paragallinarum and/or said polypeptide, variant or derivative, and/or a protective effect against Haemophilus paragallinarum infection.

Prior to testing a particular fragment for immunoreactivity in the above method, a variety of predictive methods may be used to deduce whether a particular fragment can be used to obtain an antibody that cross-reacts with the native antigen. These predictive methods may be based on amino-terminal or carboxy-terminal sequence as for example described in Chapter 11.14 of Ausubel et al., supra. Alternatively, these predictive methods may be based on predictions of hydrophilicity as for example described by Kyte & Doolittle 1982, J. Mol. Biol. 157 105 and Hopp & Woods, 1983, Mol. Immunol. 20 483) which are incorporated by reference herein, or predictions of secondary structure as for example described by Choo & Fasman,1978, Ann. Rev. Biochem. 47 251), which is incorporated herein by reference.

In addition, “epitope mapping” uses monoclonal antibodies of the invention to identify cross-reactive epitopes by first testing their ability to provide cross-protection, followed by identifying the epitope recognized by said antibodies. An exemplary method is provided in Coligan et al., CURRENT PROTOCOLS IN IMMUNOLOGY, supra.

Generally, peptide fragments consisting of 10 to 15 residues provide optimal results. Peptides as small as 6 or as large as 20 residues have worked successfully. Such peptide fragments may then be chemically coupled to a carrier molecule such as keyhole limpet haemocyanin (KLH) or bovine serum albumin (BSA) as for example described in Sections 11.14 and 11.15 of Ausubel et al., supra).

The peptides may be used to immunize an animal as for example discussed above. Antibody titers against the native or parent polypeptide from which the peptide was selected may then be determined by, for example, radioimmunoassay or ELISA as for instance described in Sections 11.16 and 114 of Ausubel et al., supra.

Antibodies may then be purified from a suitable biological fluid of the animal by ammonium sulfate fractionation or by chromatography as is well known in the art. Exemplary protocols for antibody purification are given in Sections 10.11 and 11.13 of Ausubel et al., supra, which are herein incorporated by reference.

Immunoreactivity of the antibody against the native or parent polypeptide may be determined by any suitable procedure such as, for example, by Western blot.

Functional Blockers

It is contemplated that interruption of the function of the HagA polypeptides of the invention may be of significant therapeutic benefit in diseases caused by Haemophilus paragallinarum and related bacteria, most notably infectious coryza in chickens.

The HagA polypeptides of the invention are members of the P5 group of outer membrane proteins which have been described in relation to other Haemophilus sp. It is expected that the HagA polypeptides of the invention bind or otherwise interact with molecules on chicken cells, such as in the respiratory tract.

It is therefore contemplated that moieties such as chemical reagents or polypeptides which block receptors on the cell surface which interact with a HagA polypeptide of the invention may be administered. These compete with the infective organism for receptor sites. Such moieties may comprise for example polypeptides of the invention, in particular fragments, or functional equivalents of these as well as mimetics.

The term “mimetics” is used herein to refer to chemicals that are designed to resemble particular functional regions of the proteins or peptides, and includes within its scope the terms “agonist” and “antagonist” as are well understood in the art. Anti-idiotypic antibodies raised against the above-described antibodies which antagonize binding of the bacteria to a cell surface may also be used. Alternatively, moieties which interact with the receptor binding sites in the polypeptides of the invention may effectively prevent infection of a cell by H. paragallinarum. Such moieties may comprise blocking antibodies, peptides or polypeptides derived from HagA polypeptides of the invention other chemical reagents such as carbohydrates, oligo- and poly-saccharides and small organic molecules designed to mimic receptor-binding regions of HagA polypeptides of the invention.

With regard to mimetics, computer-assisted structural database searching is becoming increasingly utilized as a procedure for identifying mimetics, such as in the form of agonists or antagonists. Database searching methods which, in principle, may be suitable for identifying mimetics of HagA polypeptides and peptides, may be found in International Publication WO 94/18232 (directed to producing HIV antigen mimetics), U.S. Pat. No. 5,752,019 and International Publication WO 97/41526 (directed to identifying EPO mimetics), each of which is incorporated herein by reference.

All such moieties, pharmaceutical compositions in which they are combined with pharmaceutically acceptable carriers and methods of treating chickens suffering from Haemophilus paragallinarum infection by administration of such moieties or compositions form a further aspect of the invention.

The HagA polypeptides of the invention may be used in the screening of compounds for their use in the above methods. For example, HagA polypeptides of the invention may be combined with a label and exposed to a cell culture in the presence of a reagent under test. The ability of reagent to inhibit the binding of the labeled polypeptide to the cell surface can then be observed. In such a screen, the labeled polypeptides may be used directly on an organism such as E. coli. Alternatively, Haemophilus paragallinarum itself may be engineered to express a modified and detectable form of the polypeptide. The use of engineered Haemophilus paragallinarum strains in this method is preferred as it is more likely that the tertiary structure of the protein will resemble more closely that expressed in wild-type bacteria.

In order that the invention may be readily understood and put into practical effect, particular preferred embodiments will now be described by way of the following non-limiting examples.

### EXAMPLE 1

### Bacterial Strains

The H. paragallinarum strains used in this study are listed in Table 2 and were obtained from Pat Blackall, Queensland Poultry Research and Development Centre, Animal Research Institute, Yeerongpilly, Queensland, Australia. Escherichia coli strain M15(pREP4) (Nal^{s}, Str^{s}, Rif^{s}, Thi⁻, Lac⁻, Ara⁺, Gal⁺, Mtl⁻, F⁻, RecA⁺, Uvr⁺, Lon⁺) was obtained from QIAGEN (Germany). E. coli SURE® cells (e14⁻, (McrA⁻) Δ (mcrCB-hsdSMR-mrr), 171 endA1, supE44, thi-1, gyrA96, relA1, lac, recB, recJ, sbcC, umuC::Tn5 (Kan^{r}, uvrC [F′, proAB, lacl^{q}Z, (M15 Tn10 (Tet^{r})]) was obtained from Stratagene (USA).

### EXAMPLE 2

### Monoclonal Antibodies

Monoclonal antibody D4 recognises a 39 kDa protein and is specific for serovar A H. paragallinarum strains. The monoclonal antibody is capable of inhibiting haemagglutination, strongly suggesting that it is directed against the H. paragallinarum haemagglutinin. MAb D4 was first described in Takagi et al., 1991b, Vet. Microbiol. 27 327 which is herein incorporated by reference.

### EXAMPLE 3

### Determination of the N-Terminal Sequence of the H. Paragallinarum Haemagglutinin Protein

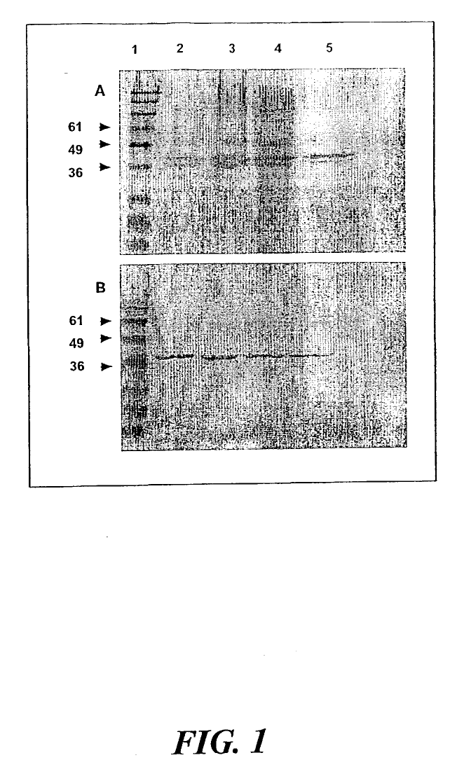

H. paragallinarum strain 0083 cells were grown overnight in liquid culture, washed several times in PBS and resuspended in 50 mM Tris-HCL, 10% glycerol, pH 8.0. Cells were lysed by sonication. The cell lysate was fractionated using ammonium sulphate to precipitate proteins at 0-20%, 20-40% and 40% ammonium sulphate concentrations. Precipitated proteins were resuspended, and a small portion of each fraction was analysed by western blotting using the MAb D4. In order to identify the HagA protein, ammonium sulphate fractionations were performed so that a well-separated band on SDS-PAGE could be identified for N-terminal sequencing. A 39 kDa protein was present in all three ammonium sulfate precipitation fractions, although it was most highly enriched in the 0-20% fraction as shown in FIG. 1. The identity of this band as the H. paragallinarum haemagglutinin antigen was confirmed by immunoblot analysis with MAb4D as shown in FIG. 1.

To enable N-terminal sequencing, the 0-20% ammonium sulfate fraction was separated by SDS-PAGE and proteins semi-purified according to molecular mass by electroelution. The eluted-haemagglutinin protein was applied to a 12% Tris-tricine polyacrylaminde gel with anode buffer (0.1 M Tris pH 8.9) and cathode buffer (0.1 M Tris, 0.1 M Tricine, 0.1% SDS at pH 8.25) and then transferred to PVDF membrane (Polyscreen PVDF Transfer Membrane, NEN™ Life Science Products, Boston, USA) using semi-dry transfer (Trans-blot semi-dry transfer cell, BIO-RAD®, USA) and CAPS buffer (10 mM CAPS, pH 11). The PVDF membrane was then soaked in Milli Q water for 10 mm with shaking, stained with 0.1% Coomassie Blue R250, 50% methanol for 5 min, destained in 50% methanol, 10% acetic acid and rinsed in Milli Q water. N-terminal sequence of the 39 kDa sized band was obtained using a PE Biosystems 492cLC protein sequencer.

The N-terminal sequence APQANTFYAGAKAG (SEQ ID NO:13) is shown in FIG. 4 and, subsequently, was shown to be present in all HagA polypeptides isolated according to the present invention.

### EXAMPLE 4

### Isolation of Haemagglutinin-Encoding Nucleic Acid by Inverse PCR

All of the primers used in nucleic acid isolation by PCR are shown in Table 3 (SEQ ID NOS:26-36).

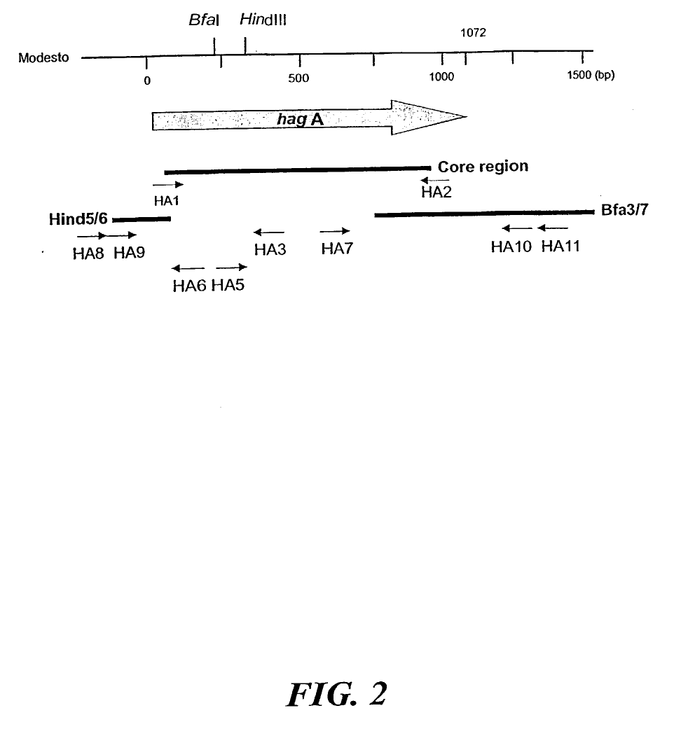

Oligonucleotide primers (HA1 and HA2; see Table 3) based on N-terminal sequence and alignments of the p5/OMP region of closely related HAP group organisms (Haemophilus, Actinobacillus and Pasturella) were used to amplify products from strains 0083, 0222 and Modesto. The strategy used is outlined in FIG. 2.

Chromosomal DNA from each of the strains was digested overnight with BfaI and HindIII restriction enzymes. Internal primers (HA3/HA7 and HA5/HA6 primer pairs) were designed to amplify either the upstream or downstream sequences of the core region (according to the position of the relevant restriction enzyme site within the core region). Restriction enzymes used to digest chromosomal DNA were heat inactivated at an appropriate temperature and the digested DNA ethanol precipitated. Ligations using two dilutions (neat and 1/100) of digested DNA were prepared using a standard protocol with T4 DNA Ligase (Promega). PCR was performed using appropriate primer pairs with a denaturation step of 94° C. for 30 sec, an annealing step of 55° C. for 30 sec and extension step of 72° C. for 3 min for 30 cycles.

Amplification of HindIII-digested strain Modesto DNA using primers HA5 and HA6 resulted in an amplification product of ˜300 bp which contained the upstream region of the gene. Primers HA3 and HA7 resulted in an amplification product of ˜1000 bp from BfaI-digested strain Modesto DNA. This amplification product revealed the downstream sequence including the stop codon. Amplification products were purified from a 1% agarose gel and sequenced. The inverse PCR products from strain Modesto were used to produce the full-length contig of the gene encoding the putative haemagglutinin polypeptide, termed hagA.

### EXAMPLE 5

### DNA Sequencing

ABI Prism™ Big Dye Primer Cycle Sequencing Ready Reaction with AmpliTaq® DNA Polymerase, FS′ (PE Applied Biosystems) was used for DNA sequencing. The samples were amplified using an Omn-E Thermal Cycler (Hybaid), with the following program: 96° C. for 10 sec, 50° C. for 5 sec and 60° C. for 4 min for 25 cycles. Following ethanol precipitation, samples were sent to Australian Genomic Research Facility (AGRF) for automated sequencing using an ABI 373A automatic sequencer (Applied Biosystems International, Perkin Elmer).

PCR primers (Table 3) were used to amplify the full-length hagA gene. The hagA gene was fully sequenced in 11 H. paragallinarum strains. Of the 11 strains, five were serotyped as Page serovar A, two were Page serovar B and four were Page serovar C (Table 2).

All of the isolated nucleic acid sequences are shown in FIG. 5 and the deduced amino acid sequences are shown in FIG. 4.

### EXAMPLE 6

### Sequence Analysis

A BLASTP search with the N-terminal sequence APQANTFYAGAKAG (SEQ ID NO:13) revealed similarity of this N-terminal sequence to the P5 gene product of Haemophilus influenzae (85% identity, 85% similarity). Various other members of the P5 family of outer membrane proteins of closely related organisms (Actinobacillus actinomycetemcomitans Omp34, Pasteurella haemolytica PomA, Haemophilus ducreyi OmpA2) belonging to the HAP group also shared close similarity with the H. paragallinarum HagA N-terminal sequence.

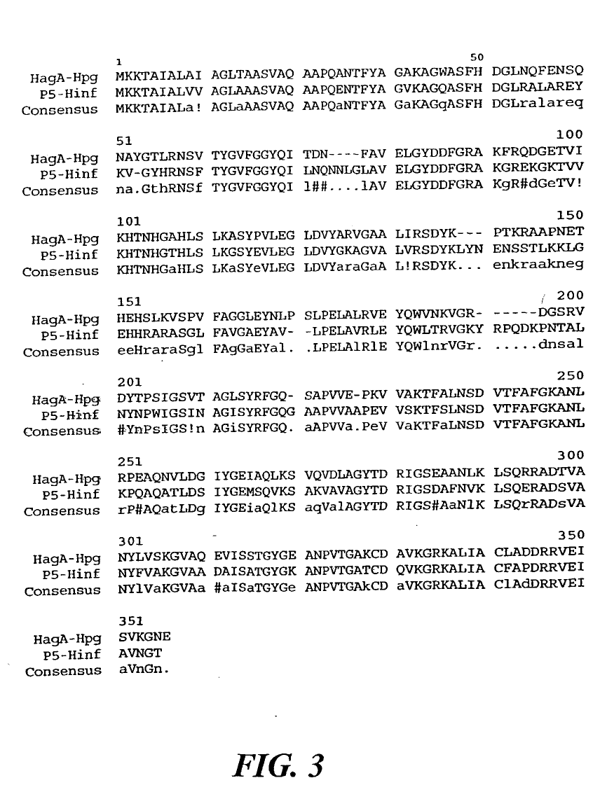

The full-length sequence of strain Modesto is 1072 bp (or 341 amino acids) in size and found to be similar using the BLASTX program (62% identity, 73% similarity) to the H. influenzae P5 gene, as shown by FIG. 3. The haemagglutinin gene (hagA) of H. paragallinarum is however slightly larger than that of its H. influenzae counterpart (1059 bp, Genbank accession number L20309) (Fleishmann et al., 1995, Science 269 496). The signal peptide cleavage site was predicted to be between the amino acid positions 21-22, as shown in FIG. 4, using the CBS SignalP program, version V1.1 (Nielsen et al., 1997, Protein Eng. 10 1). The start of the mature form of the HagA protein corresponds with the N-terminal sequence obtained (SEQ ID NO:13), as expected, and is conserved across the 11 strains sequenced.

Conserved regions present in all HagA polyeptides are shown in FIG. 4. Of the conserved regions, residues 41-50 and 131-140 are unique to all HagA polypeptides, as determined by ClustalW and BLASTP analysis.

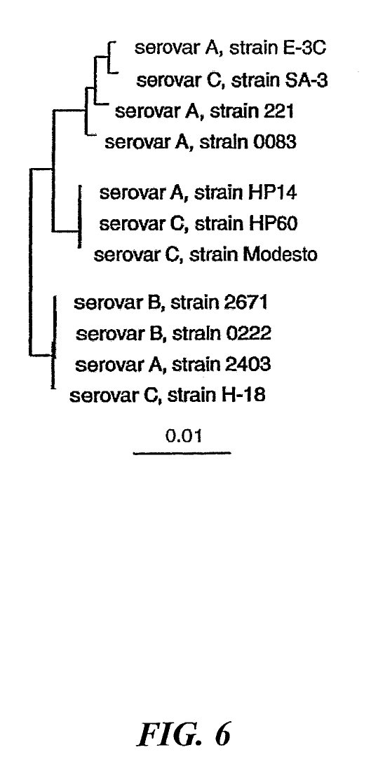

In FIG. 6, a phylogenetic tree representing the relationship between the full length hagA gene sequences of the 11 serotyping reference strains of H. paragallinarum is shown. Strains 0083, 221, 2403, E-3C, and HP14 belong to Page serovar A; strains 0222 and 2671 belong to Page serovar B and strains Modesto, H-18, SA-3 and HP60 belong to Page serovar C.

### EXAMPLE 7

### Expression, Isolation and Analysis of Recombinant H. Paragallinarum HagA Polypeptides

H. paragallinarum strain HP14 (Page serovar A) was grown on TM/SN agar (Reid & Blackall, 1987, Avian Dis. 32 59) and incubated at 37° C. overnight in the presence of 5% CO₂. A lysate was prepared by harvesting one plate of HP14 bacteria into 100 μL of sterile PBS and boiling this suspension for 10 min. The mature sequence of the hagA gene was amplified from strain HP14 using primers HA12 and HA13 (Table 3). The ˜1.1 kb PCR product was extracted using QIAQuick Gel Extraction kit (QIAGEN, Germany) and cloned into pGEM-T Easy (Promega, USA). The hagA gene was subcloned from the resulting vector into a pQE30 His-tag fusion vector (QIAGEN, Germany) by digestion with BamHI and PstI, generating pQE30hagA. This plasmid was transferred into an expression strain, E. coli M15(pREP4) by electroporation followed by selection on Luria-Bertoni (LB) Agar supplemented with 0.05% glucose, 100 μg/mL ampicillin and 25 μg/mL kanamycin. A representative clone containing the recombinant plasmid was selected for purification of rHagA. Other recombinant DNA methods used were essentially as described in Maniatis et al. Molecular Cloning: A Laboratory Manual (Cold Spring Harbour Laboratory Press, NY, 1989).

A 10 mL culture of M15(pREP4) containing pQE30/hagA was grown at 37° C. with shaking overnight in LB broth supplemented with 0.05% glucose, 100 μg/mL ampicillin and 25 μg/mL kanamycin. The overnight culture was sub-cultured into 500 mL LB broth supplemented with 0.05% glucose, 100 μg/mL ampicillin and 25 μg/mL kanamycin and grown at 37° C., with shaking to an optical density of A₆₀₀ 0.3-0.5. Expression of rHagA protein was induced at 37° C. with 0.5 mM IPTG (isopropyl-β-D-thiogalactopyranoside) for 4 hr. Cell lysis and purification of the polyhistidine-HagA fusion were as recommended in the manufacturer's instructions (QIAGEN QIA-Expressionist, Germany). Briefly, bacteria harboring pQE30hagA, were collected and centrifuged at 3500×g for 10 min at 4° C. The pellet was washed with PBS and resuspended in 50 mL denaturing lysis buffer (100 mM NaH₂PO_{4,} 10 mM Tris, 8 M Urea, pH 8.0) followed by incubation at room temperature for 1 hr with agitation. The cell debris was pelleted at 870×g for 10 min at 4° C. and supernatant bound to pre-equilibrated Ni-NTA resin (QIAGEN, Germany) for 30 min at room temperature with agitation. The Ni-NTA resin was equilibrated with 15 mL denaturing lysis buffer containing 20 mM imidazole for 30 min at room temperature with agitation. The resin was washed twice with 5 bed volumes of wash buffer (100 mM NaH₂PO_{4,} 10 mM Tris, 8 M Urea, pH 8.0, 20 mM imidazole, 500 mM NaCl). The resin was resuspended in wash buffer and packed into a 10 mL column and washed with a further 5 bed volumes of wash buffer. The His-tagged protein was eluted in three bed volumes of elution buffer (100 mM NaH₂PO_{4,} 10 mM Tris, 8 M Urea, pH 8.0, 250 mM imidazole) in 2 mL fractions. All eluted fractions were analysed by SDS-PAGE for presence of rHagA. The combined elutions containing rHagA were dialysed against PBS containing 0.05% SDS overnight at 4° C.

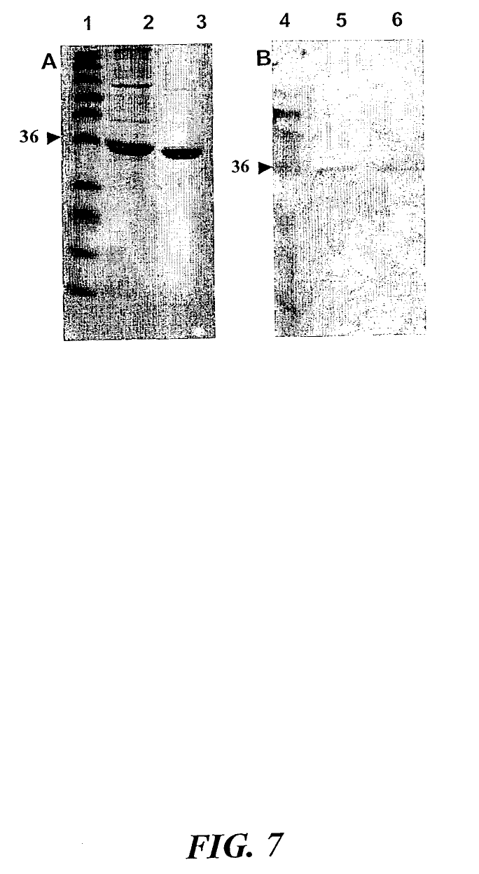

The rHagA protein purified from the Ni-NTA column was estimated to be >90% pure by SDS-PAGE analysis (FIG. 7A). From a 500 mL culture, approximately 23 mg rHagA protein was purified at a concentration of 0.58 mg/mL as determined using a BCA protein estimation kit (Pierce, USA).

Recombinant His-tagged HagA protein was analysed by immunoblot using MAb4D and the results shown in FIG. 7B. Purified rHagA was run on a 12% SDS-polyacrylamide gel, along with HP14 whole cells as a positive control. The proteins were transferred to nitrocellulose membrane (Protran®, Schleicher and Schuell, Germany) using semi-dry transfer (Trans-blot semi-dry transfer cell, BIO-RAD®, USA) according to manufacturer's instructions. MAb4D was used at a dilution of 1/50 and secondary antibody at 1/200 (Goat anti-mouse IgG-AP conjugate, Promega, USA).

### EXAMPLE 8

### Haemagglutination Assay

The assay for haemagglutination activity was performed as previously described in Blackall et al., 1990, Avian Dis. 34 643. Briefly, 50 μL of diluent was added to the appropriate wells of a U-bottomed microtiter plate. Purified rHagA protein (50 μL) was added to the first well of the row. Doubling dilutions of the purified protein were made across the plate followed by the addition of 50 μL 0.5% glutaraldehyde-fixed chicken red blood cells to each well. The plate was incubated at room temperature for 30-60 min. The haemagglutination titer was read as the highest antigen dilution giving at least 50% haemagglutination. Appropriate positive and negative controls were included in the haemagglutination assay. The positive control was a whole cell suspension of strain 0083 (Page serovar A), prepared as described previously (Blackall et al., 1990, supra). High titre hyper-immune antisera to strains 0083 and Modesto (serovars A and C, respectively) were used in a haemagglutination inhibition (HI) assay as described previously (Blackall et al., 1990, supra).

The purified recombinant protein was shown to agglutinate chicken red blood cells. A titre of 2200 HA units/mg rHagA protein was obtained. To determine whether rHagA reacted with the monoclonal antibody originally used to identify HA in H. paragallinarum cell extracts, the rHagA was analysed by immunoblot. The result shown in FIG. 7B shows reactivity of the rHagA with MAb4D. The HA activity of rHagA, in conjunction with the ability of the anti-haemagglutinin monoclonal antibody (MAb4D) to recognize the protein by immunoblot, confirms the identity of the recombinant protein as the haemagglutinin of H. paragallinarum. However, neither the MAb4D nor the high-titre polyclonal antisera to serovars A or C recognised the rHagA protein in a HI test (result not shown).

### EXAMPLE 9

### Vaccination with Purified Recombinant H. Paragallinarum HagA Polypeptides

The vaccination strategy was based on that described in Reid & Blackall, 1987, 31 59 and performed as follows.

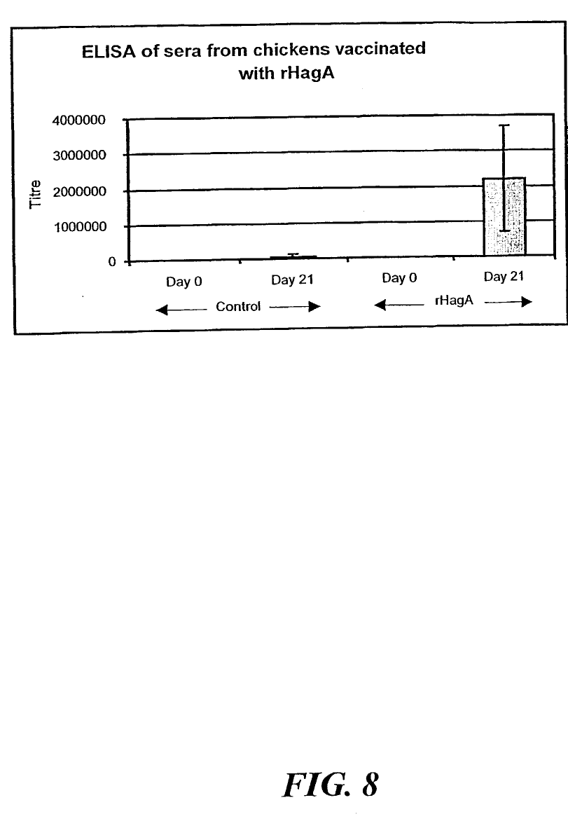

Eleven 6 week old commercial layer chickens, known to be free of H. paragallinarum infections, were vaccinated with 200 mg of recombinant HagA, protein (rHagA) in a total volume of 1 mL with incomplete Freund's adjuvant (50% adjuvant, 50% antigen). A control group of 10 birds was vaccinated with 1 mL of PBS with incomplete Freund's adjuvant (50% PBS, 50% adjuvant). Blood was collected from the birds prior to vaccination and 21 days after vaccination. To prepare serum, the blood was clotted and centrifuged at 12500×g for 2 min. Sera were stored at −20° C. until analysis. Serial two-fold dilutions of serum samples were analysed by ELISA. Plates were coated overnight at 4° C. with 100 μl of carbonate coating buffer (per L, 1.9292 g Sodium carbonate, 3.8052 g Sodium hydrogen carbonate, pH 9.6) containing 10 μg/ml rHagA. Plates were blocked for 2 hrs at 37° C. with 5% skim milk in PBS (150 μL/well). 100 μl aliquots of serum (in 0.5% skim milk in PBS) was added to each well, and incubated at 37° C. for 1 hr. Plates were washed six times with PBS/0.5% Tween 20. After washing plates were incubated with 100 μL/well secondary antibody (Affinity purified peroxidase labelled Goat anti-Chicken IgG (H+L), Kirkegaard & Perry Laboratories, USA, in 0.5% skim milk/PBS) at 37° C. for 1 hr, followed by washing three times with PBS/0.5% Tween 20. Assays were developed for 30 min at room temperature with 100 μL/well ABTS solution per well (200 μl ABTS stock (280 mg ABTS/10 mL water), 200 μL hydrogen peroxide solution (126 μL/10 mL water), 10 mL substrate buffer (substrate buffer=200 mM Na₂HPO₄ adjusted to pH 4.2 using 100 mM citric acid). Then the aborbance of each well at 405 nm was determined. The results are shown in FIG. 8 where titre is expressed as the reciprocal of the last dilution which showed reactivity. No significant differences between the titres of the control and rHagA groups prior to vaccination at day 0 were observed (p=0.4328). However, 21 days after vaccination, a highly significant increase in anti-rhagA titre was observed in the rhagA group when compared to the controls (p=0.0005).

### EXAMPLE 10

### Vaccination with Recombinant H. Paragallinarum HagA Polypeptides Expressed in a Live Attenuated Vector

Live attenuated Salmonella expressing the HagA polypeptide under the control of an inducible promoter will be constructed. Cultures of the Salmonella strain will be grown, and the resulting cells washed with phosphate buffered saline. The resulting cell suspension will be inoculated intranasally or orally into chickens as a dose sufficient to allow colonization of the chicken. Vaccination may be repeated after 14 days with immune responses being measured regularly. The immune response will be monitored by ELISA or Western blotting to detect antibodies against H. paragallinarum or Salmonella, or against HagA polypeptides. A control group, consisting of birds vaccinated with Salmonella that have not been modified to express the haemagglutinin will be included. One to 3 weeks after the final vaccination chickens will be challenged with a virulent H. paragallinarum strain. Chickens will be monitored for 7 days before necroscopy. Vaccine efficacy will be monitored using clinical signs (over 7 days), and gross pathology and comparative reisolation rates of H. paragallinarum.

Throughout this specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All patent and scientific literature, computer programs and algorithms referred to in the specification are incorporated herein by reference.

TABLE 1

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gin |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Vai | Ile, Leu |

TABLE 2

| | Source | Page | Kume | PCR |
|---|---|---|---|---|
| Strain^{a} | Country | Serovar | Serovar | Primer Set |
| 0083 (P) | USA | A | A-1 | (2) |
| 0222 (P) | USA | B | B-1 | (1) |
| Modesto (K,P) | USA | C | C-2 | (2) |
| 221 (K) | Japan | A | A-1 | (2) |
| 2403 (K) | Germany | A | A-2 | (1) |
| E-3C (K) | Brazil | A | A-3 | (1) |
| HP14 (K) | Australia | A | A-4 | (2) |
| 2671 (K) | Germany | B | B-1 | (1) |
| H-18 (K) | Japan | C | C-1 | (1) |
| SA-3 (K) | South | C | C-3 | (1) |
| | Africa | | | |
| HP60 (K) | Australia | C | C-4 | (1) |

TABLE 3

| | |
|---|---|
| Primer | Sequence (5′→3′) |

| | | |
|---|---|---|
| HA1 | TGTAGCTCAAGCAGCTCCACAAG | (SEQ ID NO:26) |
| HA2 | TCAAGCGATAAGTGCTTTACGACC | (SEQ ID NO:27) |
| HA3 | AACGCGAGCATAAACATC | (SEQ ID NO:28) |
| HA5 | GCTGTTGAGCTAGGTTA | (SEQ ID NO:29) |
| HA6 | AGATGCCCAGCCCGCTT | (SEQ ID NO:30) |
| HA7 | CGGTTCTGTAACTGCTGG | (SEQ ID NO:31) |
| HA8 | AAGCTTTTATTTTAGATTTATTG | (SEQ ID NO:32) |
| HA10 | CTGCTTGCACTAAGCCGTTG | (SEQ ID NO:33) |
| HA11 | CGCACGGCATTGATATTGTG | (SEQ ID NO:34) |
| HA12 | CGCGGATCCGCACCACAAGCAAATACTTTC | (SEQ ID NO:35) |
| HA13 | TGCAGACGTCAACGTGTTAAGAAATTACTCG | (SEQ ID NO:36) |

1. An isolated polypeptide comprising an amino acid sequence defined by residues 41-50 of any one of SEQ ID NOS:1-12.

2. An isolated polypeptide comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

3. A mature form of the isolated polypeptide of claim 2 having an N-terminus defined by an amino acid sequence set forth in SEQ ID NO:13.

4. The isolated polypeptide of claim 1, which is a recombinant haemagglutinin polypeptide of Haemophilus paragallinarum.

5. A biologically-active fragment, variant or derivative of the isolated polypeptide of claim 2, wherein the biologically-active fragment, variant or derivative is selected from the group consisting of:
(i) a mature form of said isolated polypeptide having haemagglutinin activity;
(ii) an immunogen which is capable of eliciting an immune response in an avian; and
(iii) an immunogen which is capable of eliciting an immune response that provides protection against one or more strains of Haemophilus paragallinarum in a chicken.

6. The isolated polypeptide of claim 1 which is capable of eliciting an immune response in an avian.

7. The isolated polypeptide of claim 6, wherein said immune response provides protection against one or more strains of Haemophilus paragallinarum in a chicken.

8. A pharmaceutical composition comprising at least one isolated polypeptide according to claim 1 together with a pharmaceutically-acceptable carrier, diluent or excipient.

9. The pharmaceutical composition of claim 8 which is a vaccine.

10. An isolated nucleic acid that encodes an isolated polypeptide according to claim 1.

11. The isolated nucleic acid of claim 10, which comprises residues 121-150 of any one of SEQ ID NOS:15-25.

12. The isolated nucleic acid of claim 11, further comprising the nucleotide sequence set forth in SEQ ID NO:14.

13. The isolated nucleic acid of claim 12 comprising a nucleotide sequence selected from the group consisting of: SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24 and SEQ ID NO:25.

14. An isolated nucleic acid homolog of the isolated nucleic acid of claim 10.

15. An expression construct comprising the isolated nucleic acid of claim 10 or the homolog of claim 14.

16. A host cell transformed with the expression construct of claim 15.

17. The host cell of claim 16, which is a bacterium.

18. The host cell of claim 17, which is Salmonella or Mycoplasma.

19. The host cell of claim 17, which is attenuated.

20. A vaccine comprising the host cell of claim 19 together with a pharmaceutically-acceptable carrier, diluent or excipient.

21. A method of immunizing an avian, said method including the step of administering at least one isolated polypeptide according to claim 1 to said avian.

22. The method of claim 21, wherein the at least one isolated polypeptide is administered as a pharmaceutical composition comprising a pharmaceutically-acceptable carrier, diluent or excipient.

23. A method of immunizing an avian, said method including the step of administering at least one isolated nucleic acid according to claim 10 to said animal.

24. The method of claim 23, wherein the at least one isolated nucleic acid is administered in an attenuated bacterium.

25. The method of claim 24, wherein the attenuated bacterium is Salmonella or Mycoplasma.

26. A method of isolating a nucleic acid homolog, said method comprising the step of using one or more primers and a nucleotide sequence amplification technique to produce an amplification product corresponding to said nucleic acid homolog, wherein the one or more primers are derived from a nucleotide sequence selected from the group consisting of: SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24 and SEQ ID NO:25.

27. The method of claim 26, wherein the nucleotide sequence amplification technique is PCR.

28. The method of claim 26, wherein the isolated nucleic acid, homolog or fragment thereof is isolated from a nucleic acid sample obtained from an avian.

29. A method of detecting H. paragallinarum including the step of detecting an isolated nucleic acid, homolog or fragment thereof, wherein the isolated nucleic acid comprises a nucleotide sequence selected from the group consisting of: SEQ ID NO:14, SEQ ID NO:15, SEQ ID NO:16, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24; SEQ ID NO:25.

30. The method of claim 29, wherein the detection step uses PCR.

31. The method of claim 30, wherein the detection step uses one or more primers comprising a nucleotide sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36.

32. The method of claim 29, wherein the isolated nucleic acid, homolog or fragment thereof is detected in a nucleic acid sample obtained from an avian.

33. A method of identifying an immunogenic fragment of the isolated polypeptide of claim 1 including the steps of:
(i) producing a fragment of said polypeptide, variant or derivative;
(ii) administering said fragment to a mammal or an avian; and
(iii) detecting an immune response in said mammal or avian, which response includes production of elements which specifically bind H. paragallinarum and/or said polypeptide, variant or derivative, and/or a protective effect against H. paragallinarum infection.


SEQUENCE LISTING (39 sequences)

SEQ ID NO: 1 - protein, 344 aa, Haemophilus paragallinarum
MKKTAIALVIAGLTAASVAQAAPQANTFYAGAKAGWASFHDGLNQFENSQNAYGTLRNSVTYGVFGGYQITDNFAVELGYDDFGRAKLRQDGETVGKHTNHGAHLSLKASYPVLEGLDVYARVGAALIRSDYKPTKRAAPNETHEHSLKVSPVFAGGLEYNLPSLPELALRVEYQWVNKVGRWEKDGSRVDYTPSIGSVTAGLSYRFGQSAPVVEPKVVAKTFALNSDVTFAFGKANLRPEAQNVLDGIYGEIAQLKSVQVDLAGYTDRIGSEAANLKLSQRRADTVANYLVSKGVAQEVISSTGYGEANPVTGAKCDAVKGRKALIACLADDRRVEISVKGNE

SEQ ID NO: 2 - protein, 344 aa, Haemophilus paragallinarum; identical to SEQ ID NO: 1

SEQ ID NO: 3 - protein, 344 aa, Haemophilus paragallinarum
MKKTAIALAIAGLTAASVAQAAPQANTFYAGAKAGWASFHDGLNQFENSQNADGTLRNSVTYGVFGGYQITDNFAVELGYDDFGRAKSRQGGETVIKHTNHGAHLSLKASYPVLEGLDVYARVGAALIRSDYKPTKRAAPNQTHEHSLKVSPVFAGGLEYNLPSLPELALRVEYQWVNKVGRVEKDGSRVDYTPSIGSVTAGLSYRFGQSAPVVEPKVVAKTFALNSDVTFAFGKANLRPEAQNVLDGIYGEIAQLKSVQVDVAGYTDRIGSEAANLKLSQRRADTVANYLVSKGVAQEVISSTGYGEANPVTGAKCDTVKGRKALIACLADDRRVEISVKGEE

SEQ ID NO: 4 - protein, 344 aa, Haemophilus paragallinarum; identical to SEQ ID NO: 1

SEQ ID NO: 5 - protein, 341 aa, Haemophilus paragallinarum
MKKTAIALAIAGLTAASVAQAAPQANTFYAGAKAGWASFHDGLNQFENSQNAYGTLRNSVTYGVFGGYQITDNFAVELGYDDFGRAKFRQDGETVIKHTNHGAHLSLKASYPVLEGLDVYARVGAALIRSDYKPTKRAAPNETHEHSLKVSPVFAGGLEYNLPSLPELALRVEYQWVNKVGRDGSRVDYTPSIGSVTAGLSYRFGQSAPVVEPKVVAKTFALNSDVTFAFGKANLRPEAQNVLDGIYGEIAQLKSVQVDLAGYTDRIGSEAANLKLSQRRADTVANYLVSKGVAQEVISSTGYGEANPVTGAKCDAVKGRKALIACLADDRRVEISVKGNE

SEQ ID NO: 6 - protein, 344 aa, Haemophilus paragallinarum
MKKTAIALAIAGLTAASVAQAAPQANTFYAGAKAGWASFHDGLNQFENSQNADGTLRNSVTYGVFGGYQITDNFAVELGYDDFGRAKRRQGGETVIKYTNHGAHLSLKASYPVLEGLDVYARVGAALIRSDYKPTKRAAPNQTHEHSLKVSPVFAGGLEYNLPSLPELALRVEYQWVNKVGRVEKDGSRVDYTPSIGSVTAGLSYRFGQSAPVVEPKVVAKTFALNSDVTFAFGKANLRPEAQNVLDGIYGEIAQLKSVQVDVAGYTDRIGSEAANLKLSQRRADTVANYLVSKGVAQEVISSTGYGEANPVTGAKCDTVKGRKALIACLADDRRVEISVKGEE

SEQ ID NO: 7 - protein, 344 aa, Haemophilus paragallinarum
MKKTAIALAIAGLTAASVAQAAPQANTFYAGAKAGWASFHDGLNQFENSQNADGTLRNSVTYGVFGGYQITDNFAVELGYDDFGRAKRRQGGETVIKHTNHGAHLSLKASYPVLEGLDVYARVGAALIRSDYKPTKRAAPNQTHEHSLKVSPVFAGGLEYNLPSLPELALRVEYQWVNKVGRVEKDGSRVDYTPSIGSVTAGLSYRFGQSAPVVEPKVVAKTFALNSDVTFAFGKANLRPEAQNVLDGIYGEIAQLKSVQVDVAGYTDRIGSEAANLKLSQRRADTVANYLVSKGVAQEVISSTGYGEANPVTGAKCDTVKGRKALIACLADDRRVEISVKGEE

SEQ ID NO: 8 - protein, 341 aa, Haemophilus paragallinarum; identical to SEQ ID NO: 5

SEQ ID NO: 9 - protein, 344 aa, Haemophilus paragallinarum
MKKTAIALAIAGLTAASVAQAAPQANTFYAGAKAGWASFHDGLNQFENSQNADGTLRNSVTYGVFGGYQITDNFAVELGYDDFGRAKLRQGGETVIKYTNHGAHLSLKASYPVLEGLDVYARVGAALIRSDYKPTKRAAPNQTHEHSLKVSPVFAGGLEYNLPSLPELALRVEYQWVNKVGRVEKDGSRVDYTPSIGSVTAGLSYRFGQSAPVVEPKVVAKTFALNSDVTFAFGKANLRPEAQNVLDGIYGEIAQLKSVQVDVAGYTDRIGSEAANLKLSQRRADTVANYLVSKGVAQEVISSTGYGEANPVTGAKCDTVKGRKALIACLADDRRVEISVKGEE

SEQ ID NO: 10 - protein, 344 aa, Haemophilus paragallinarum
MKKTAIALVIAGLTAASVAQAAPQANTFYAGAKAGWASFHDGLNQFENSQNAYGTLRNSVTYGVFGGYQITDNFAVELGYDDFGRAKLRQDGETVGKHTNHGAHLSLKASYPVLEGLDVYARVGAALIRSDYKPTKRAAPNETHEHTLKVSPVFAGGLEYNLPSLPELALRVEYQWVNKVGRWEKDGSRVDYTPSIGSVTAGLSYRFGQSAPVVEPKVVAKTFALNSDVTFAFGKANLRPEAQNVLDGIYGEIAQLKSVQVDLAGYTDRIGSEAANLKLSQRRADTVANYLVSKGVAQEVISSTGYGEANPVTGAKCDAVKGRKALIACLADDRRVEISVKGNE

SEQ ID NO: 11 - protein, 341 aa, Haemophilus paragallinarum; identical to SEQ ID NO: 5

SEQ ID NO: 12 - protein, 344 aa, Artificial Sequence
  Description of Artificial Sequence Consensus sequence
MKKTAIALXIAGLTAASVAQAAPQANTFYAGAKAGWASFHDGLNQFENSQNAXGTLRNSVTYGVFGGYQITDNFAVELGYDDFGRAKXRQXGETVXKXTNHGAHLSLKASYPVLEGLDVYARVGAALIRSDYKPTKRAAPNXTHEHSLKVSPVFAGGLEYNLPSLPELALRVEYQWVNKVGRXXXDGSRVDYTPSIGSVTAGLSYRFGQSAPVVEPKVVAKTFALNSDVTFAFGKANLRPEAQNVLDGIYGEIAQLKSVQVDXAGYTDRIGSEAANLKLSQRRADTVANYLVSKGVAQEVISSTGYGEANPVTGAKCDXVKGRKALIACLADDRRVEISVKGXE

SEQ ID NO: 13 - protein, 14 aa, Haemophilus paragallinarum
APQANTFYAGAKAG

SEQ ID NO: 14 - DNA, 42 bp, Haemophilus paragallinarum
gcaccacaagcaaayactttctatgctggtgcaaaagcgggc

SEQ ID NO: 15 - DNA, 1035 bp, Haemophilus paragallinarum
atgaaaaaaactgcaatcgcattagtaatcgctggtttaactgccgcgtcagtagcacaagctgcaccacaagcaaatactttctatgctggtgcaaaagcgggctgggcatctttccacgatggtttaaaccaatttgaaaactcacaaaatgcatatggtacattgcgtaattctgtaacttatggggtgtttggtggttatcaaattactgataattttgctgttgagctaggttatgatgactttggacgtgcgaaacttcgtcaagacggtgaaactgttggaaaacatacaaatcacggggctcatttaagcttaaaagcaagttatccagtgcttgaaggattagatgtttatgctcgcgttggagctgcgttaattcgttctgattataaaccaactaaaagagcagcccctaatgagacgcacgaacatagcttaaaagtttctccagtgtttgctggtggcttagagtataacttaccatcattaccagaacttgcattacgtgttgaatatcaatgggtgaataaagtagggcgttgggaaaaagatggtagccgtgtagattatacaccaagcatcggttctgtaactgctggtttatcttaccgttttggccaaagtgcaccagttgttgaacctaaggttgttgcaaaaacatttgcattaaattcagacgttactttcgcatttggtaaagcaaatttacgtccagaagcacaaaatgtattagacggtatttatggtgaaatcgcacagttaaaatcagtacaagtagatcttgctggttatactgaccgtattggtagcgaagcagccaacttaaaattatcacaacgtcgtgctgacactgtggctaactacttagtttctaaaggtgttgctcaagaagtgatttcttcaacaggttatggtgaagcgaacccagtaactggtgcgaaatgtgatgcggttaaaggtcgcaaagcattaatcgcttgtttagcagacgatcgtcgtgtagaaatctcagttaaaggtaacgagtaa

SEQ ID NO: 16 - DNA, 1035 bp, Haemophilus paragallinarum; identical to SEQ ID NO: 15

SEQ ID NO: 17 - DNA, 1035 bp, Haemophilus paragallinarum
atgaaaaaaactgcaatcgcattagcaatcgctggtttaactgctgcgtcagtagcacaagctgcaccacaagcaaacactttctatgctggtgcaaaagcgggctgggcatctttccacgatggtttaaaccaatttgaaaactcacaaaatgcagatggtacattgcgtaattctgtaacttatggggtgttcggtggttaccaaattactgataacttcgctgttgagctaggttatgatgactttgggcgtgctaaaagccgtcaaggcggtgaaactgttataaaacacacaaatcacggagctcacttaagcttaaaagcaagttatccagtgcttgaaggattagatgtttatgctcgcgttggagcagcgttgattcgttctgattataaaccaactaaaagagcagctcctaatcagacgcacgaacatagcttaaaagtttctccagtattcgctggtggtttagagtataacttaccatcattaccagaacttgcattacgtgttgaatatcaatgggtaaataaagtaggacgtgtggaaaaagatggtagccgtgtagattatacaccaagcatcggttctgtaactgctggtttatcttaccgttttggtcaaagtgcaccagttgttgaacctaaggttgttgcaaaaacatttgcattaaattcagatgttactttcgcatttggtaaagcaaatttacgtccagaagcacaaaatgtattagacggtatttatggtgaaatcgcacagttaaaatcagtacaagtagatgttgctggttatactgaccgtattggtagcgaagcagccaacttgaaattatcacaacgtcgtgctgatactgtggctaactacttagtttctaaaggtgttgctcaagaagtgatttcttcaacaggttatggtgaagcgaacccagtaactggtgcgaaatgtgatacggttaaaggtcgcaaagcattaatcgcttgtttagcagacgatcgtcgtgtagaaatctcagttaaaggtgaagagtaa

SEQ ID NO: 18 - DNA, 1035 bp, Haemophilus paragallinarum; identical to SEQ ID NO: 15

SEQ ID NO: 19 - DNA, 1026 bp, Haemophilus paragallinarum
atgaaaaaaactgcaatcgcattagcaatcgctggtttaactgctgcgtcagtagcacaagctgcaccacaagcaaacactttctatgctggtgcaaaagcgggctgggcatctttccacgatggtttaaaccaatttgaaaactcacaaaatgcatatggtacattgcgtaattctgtaacttatggtgtgtttggtggttaccaaattactgataacttcgctgttgagctaggttatgatgactttggacgtgcgaaattccgccaagacggtgaaactgttataaaacatacaaatcacggggctcacttaagcttaaaagcaagttatccagtgcttgaggggttagatgtttatgctcgcgttggagcagcattgattcgttctgattataaaccaactaaaagagcagcccctaatgagacgcacgaacacagcttaaaagtatctccagtatttgcaggaggtttagagtataacttaccatcattaccagaacttgcattacgtgttgaatatcaatgggtaaataaagtaggacgtgatggtagccgtgtagattatacaccaagcatcggttctgtaactgctggtttatcttaccgttttggtcaaagtgcaccagttgttgaacctaaggttgttgcaaaaacatttgcattaaattcagatgttactttcgcatttggtaaagcaaatttacgtccagaagcacaaaatgtattagacggtatttatggtgaaatcgcacagttaaaatcagtacaagtagatcttgctggttatactgaccgtattggtagcgaagcagccaacttaaaattatcacaacgtcgtgctgacactgtggctaactacttagtttctaaaggtgttgctcaagaagtgatttcttcaacaggttatggtgaagcgaacccagtaactggtgcgaaatgtgatgcggttaaaggtcgcaaagcattaatcgcttgtttagcagacgatcgtcgtgtagaaatctcagttaaaggtaacgagtaa

SEQ ID NO: 20 - DNA, 1035 bp, Haemophilus paragallinarum
atgaaaaaaactgcaatcgcattagcaatcgctggtttaactgctgcgtcagtagcacaagctgcaccacaagcaaacactttctatgctggtgcaaaagcgggctgggcatctttccacgatggtttaaaccaatttgaaaactcacaaaatgcagatggtacattgcgtaattctgtaacttatggggtgttcggtggttaccaaattactgataacttcgctgttgagctaggttatgatgactttgggcgtgctaaacgccgtcaaggcggtgaaactgttataaaatacacaaatcacggagctcacttaagcttaaaagcaagttatccagtgcttgaaggattagatgtttatgctcgcgttggagcagcgttgattcgttctgattataaaccaactaaaagagcagctcctaatcagacgcacgaacatagcttaaaagtttctccagtattcgctggtggtttagagtataacttaccatcattaccagaacttgcattacgtgttgaatatcaatgggtaaataaagtaggacgtgtggaaaaagatggtagccgtgtagattatacaccaagcatcggttctgtaactgctggtttatcttaccgttttggtcaaagtgcaccagttgttgaacctaaggttgttgcaaaaacatttgcattaaattcagatgttactttcgcatttggtaaagcaaatttacgtccagaagcacaaaatgtattagacggtatttatggtgaaatcgcacagttaaaatcagtacaagtagatgttgctggttatactgaccgtattggtagcgaagcagccaacttgaaattatcacaacgtcgtgctgatactgtggctaactacttagtttctaaaggtgttgctcaagaagtgatttcttcaacaggttatggtgaagcgaacccagtaactggtgcgaaatgtgatacggttaaaggtcgcaaagcattaatcgcttgtttagcagacgatcgtcgtgtagaaatctcagttaaaggtgaagagtaa

SEQ ID NO: 21 - DNA, 1035 bp, Haemophilus paragallinarum
atgaaaaaaactgcaatcgcattagcaatcgctggtttaactgctgcgtcagtagcacaagctgcaccacaagcaaacactttctatgctggtgcaaaagcgggctgggcatctttccacgatggtttaaaccaatttgaaaactcacaaaatgcagatggtacattgcgtaattctgtaacttatggggtgttcggtggttaccaaattactgataacttcgctgttgagctaggttatgatgactttgggcgtgctaaacgccgtcaaggcggtgaaactgttataaaacacacaaatcacggagctcacttaagcttaaaagcaagttatccagtgcttgaaggattagatgtttatgctcgcgttggagcagcgttgattcgttctgattataaaccaactaaaagagcagctcctaatcagacgcacgaacatagcttaaaagtttctccagtattcgctggtggtttagagtataacttaccatcattaccagaacttgcattacgtgttgaatatcaatgggtaaataaagtaggacgtgtggaaaaagatggtagccgtgtagattatacaccaagcatcggttctgtaactgctggtttatcttaccgttttggtcaaagtgcaccagttgttgaacctaaggttgttgcaaaaacatttgcattaaattcagatgttactttcgcatttggtaaagcaaatttacgtccagaagcacaaaatgtattagacggtatttatggtgaaatcgcacagttaaaatcagtacaagtagatgttgctggttatactgaccgtattggtagcgaagcagccaacttgaaattatcacaacgtcgtgctgatactgtggctaactacttagtttctaaaggtgttgctcaagaagtgatttcttcaacaggttatggtgaagcgaacccagtaactggtgcgaaatgtgatacggttaaaggtcgcaaagcattaatcgcttgtttagcagacgatcgtcgtgtagaaatctcagttaaaggtgaagagtaa

SEQ ID NO: 22 - DNA, 1026 bp, Haemophilus paragallinarum; identical to SEQ ID NO: 19

SEQ ID NO: 23 - DNA, 1035 bp, Haemophilus paragallinarum
atgaaaaaaactgcaatcgcattagcaatcgctggtttaactgctgcgtcagtagcacaagctgcaccacaagcaaacactttctatgctggtgcaaaagcgggctgggcatctttccacgatggtttaaaccaatttgaaaactcacaaaatgcagatggtacattgcgtaattctgtaacttatggggtgttcggtggttaccaaattactgataacttcgctgttgagctaggttatgatgactttgggcgtgctaaactccgtcaaggcggtgaaactgttataaaatacacaaatcacggagctcacttaagcttaaaagcaagttatccagtgcttgaaggattagatgtttatgctcgcgttggagcagcgttgattcgttctgattataaaccaactaaaagagcagctcctaatcagacgcacgaacatagcttaaaagtttctccagtattcgctggtggtttagagtataacttaccatcattaccagaacttgcattacgtgttgaatatcaatgggtaaataaagtaggacgtgtggaaaaagatggtagccgtgtagattatacaccaagcatcggttctgtaactgctggtttatcttaccgttttggtcaaagtgcaccagttgttgaacctaaggttgttgcaaaaacatttgcattaaattcagatgttactttcgcatttggtaaagcaaatttacgtccagaagcacaaaatgtattagacggtatttatggtgaaatcgcacagttaaaatcagtacaagtagatgttgctggttatactgaccgtattggtagcgaagcagccaacttgaaattatcacaacgtcgtgctgatactgtggctaactacttagtttctaaaggtgttgctcaagaagtgatttcttcaacaggttatggtgaagcgaacccagtaactggtgcgaaatgtgatacggttaaaggtcgtaaagcattaatcgcttgtttagcagacgatcgtcgtgtagaaatctcagttaaaggtgaagagtaa

SEQ ID NO: 24 - DNA, 1035 bp, Haemophilus paragallinarum
atgaaaaaaactgcaatcgcattagtaatcgctggtttaactgccgcgtcagtagcacaagctgcaccacaagcaaatactttctatgctggtgcaaaagcgggctgggcatctttccacgatggtttaaaccaatttgaaaactcacaaaatgcatatggtacattgcgtaattctgtaacttatggggtgtttggtggttatcaaattactgataattttgctgttgagctaggttatgatgactttggacgtgcgaaacttcgtcaagacggtgaaactgttggaaaacatacaaatcacggggctcatttaagcttaaaagcaagttatccagtgcttgaaggattagatgtttatgctcgcgttggagctgcgttaattcgttctgattataaaccaactaaaagagcagcccctaatgagacgcacgaacataccttaaaagtttctccagtgtttgctggtggcttagagtataacttaccatcattaccagaacttgcattacgtgttgaatatcaatgggtgaataaagtagggcgttgggaaaaagatggtagccgtgtagattatacaccaagcatcggttctgtaactgctggtttatcttaccgttttggccaaagtgcaccagttgttgaacctaaggttgttgcaaaaacatttgcattaaattcagacgttactttcgcatttggtaaagcaaatttacgtccagaagcacaaaatgtattagacggtatttatggtgaaatcgcacagttaaaatcagtacaagtagatcttgctggttatactgaccgtattggtagcgaagcagccaacttaaaattatcacaacgtcgtgctgacactgtggctaactacttagtttctaaaggtgttgctcaagaagtgatttcttcaacaggttatggtgaagcgaacccagtaactggtgcgaaatgtgatgcggttaaaggtcgcaaagcattaatcgcttgtttagcagacgatcgtcgtgtagaaatctcagttaaaggtaacgagtaa

SEQ ID NO: 25 - DNA, 1026 bp, Haemophilus paragallinarum; identical to SEQ ID NO: 19

SEQ ID NO: 26 - DNA, 23 bp, Artificial Sequence
  Description of Artificial Sequence Primer
tgtagctcaagcagctccacaag

SEQ ID NO: 27 - DNA, 24 bp, Haemophilus paragallinarum
tcaagcgataagtgctttacgacc

SEQ ID NO: 28 - DNA, 18 bp, Artificial Sequence
  Description of Artificial Sequence Primer
aacgcgagcataaacatc

SEQ ID NO: 29 - DNA, 17 bp, Artificial Sequence
  Description of Artificial Sequence Primer
gctgttgagctaggtta

SEQ ID NO: 30 - DNA, 17 bp, Artificial Sequence
  Description of Artificial Sequence Primer
agatgcccagcccgctt

SEQ ID NO: 31 - DNA, 18 bp, Artificial Sequence
  Description of Artificial Sequence Primer
cggttctgtaactgctgg

SEQ ID NO: 32 - DNA, 23 bp, Artificial Sequence
  Description of Artificial Sequence Primer
aagcttttattttagatttattg

SEQ ID NO: 33 - DNA, 20 bp, Artificial Sequence
  Description of Artificial Sequence Primer
ctgcttgcactaagccgttg

SEQ ID NO: 34 - DNA, 20 bp, Artificial Sequence
  Description of Artificial Sequence Primer
cgcacggcattgatattgtg

SEQ ID NO: 35 - DNA, 30 bp, Artificial Sequence
  Description of Artificial Sequence Primer
cgcggatccgcaccacaagcaaatactttc

SEQ ID NO: 36 - DNA, 31 bp, Artificial Sequence
  Description of Artificial Sequence Primer
tgcagacgtcaacgtgttaagaaattactcg

SEQ ID NO: 37 - protein, 352 aa, Haemophilus influenzae
MKKTAIALVVAGLAAASVAQAAPQENTFYAGVKAGQASFHDGLRALAREYKVGYHRNSFTYGVFGGYQILNQNNLGLAVELGYDDFGRAKGREKGKTVVKHTNHGTHLSLKGSYEVLEGLDVYGKAGVALVRSDYKLYNENSSTLKKLGEHHRARASGLFAVGAEYAVLPELAVRLEYQWLTRVGKYRPQDKPNTALNYNPWIGSINAGISYRFGQGAAPVVAAPEVVSKTFSLNSDVTFAFGKANLKPQAQATLDSIYGEMSQVKSAKVAVAGYTDRIGSDAFNVKLSQERADSVANYFVAKGVAADAISATGYGKANPVTGATCDQVKGRKALIACFAPDRRVEIAVNGT

SEQ ID NO: 38 - protein, 356 aa, Artificial Sequence
  Description of Artificial Sequence Consensus sequence
MKKTAIALXXAGLXAASVAQAAPQXNTFYAGXKAGXASFHDGLXXXXXXXXXXGXXRNSXTYGVFGGYQIXXXXXXXXAVELGYDDFGRAKXRXXGXTVXKHTNHGXHLSLKXSYXVLEGLDVYXXXGXALXRSDYKXXXXXXXXXXXXXXXHXXXXSXXFAXGXEYXXXXLPELAXRXEYQWXXXVGXXXXXXXXXXXXXYXPXIGSXXAGXSYRFGQXXAPVVXXPXVVXKTFXLNSDVTFAFGKANLXPXAQXXLDXIYGEXXQXKSXXVXXAGYTDRIGSXAXNXKLSQXRADXVANYXVXKGVAXXXISXTGYGXANPVTGAXCDXVKGRKALIACXAXDRRVEIXVXGXX

SEQ ID NO: 39 - protein, 6 aa, Artificial Sequence
  Description of Artificial Sequence 6-His tag
HHHHHH